(19) United States
(12) Patent Application Publication
(10) Pub. No.: US 2025/0127720 A1
(43) Pub. Date: Apr. 24, 2025

(54) IONIZABLE LIPID NANOPARTICLES FOR IN UTERO mRNA DELIVERY

(71) Applicant: William PERANTEAU, Philadelphia, PA (US)

(72) Inventor: William PERANTEAU, Philadelphia, PA (US)

(73) Assignee: William PERANTEAU, Philadelphia, PA (US)

(21) Appl. No.: 18/815,064

(22) Filed: Aug. 26, 2024

Related U.S. Application Data
(63) Continuation of application No. 18/261,181, filed on Jan. 1, 0001.
(60) Provisional application No. 63/136,549, filed on Jan. 12, 2021.

(51) Int. Cl. A61K 9/1271; A61K 9/00; A61K 9/51; A61K 31/7105; A61K 47/69; B82Y 5/00

(52) U.S. Cl. CPC A61K 9/1271; A61K 9/0019; A61K 9/5123; A61K 31/7105; A61K 47/6913; B82Y 5/00

(57) ABSTRACT

Disclosed herein is a method for the delivery of prenatal therapeutics, enzyme replacement therapy, or gene therapy to a fetus in need thereof. The method comprises introducing ionizable lipid nanoparticles (LNPs) nanoparticles comprising a therapeutic mRNA composition into the circulation of the fetus in need of treatment such that the ionizable LNPs deliver the therapeutic mRNA composition.

### CROSS-REFERENCE TO RELATED APPLICATIONS

This application is a Continuation of U.S. application Ser. No. 18/261,181, filed on Jul. 12, 2023, which is a national phase of PCT International Application No. PCT/US2022/012109, filed on Jan. 12, 2022, which claims priority to U.S. Application No. 63/136,549 filed Jan. 12, 2021, which are hereby incorporated by reference in their entirety.

### FIELD OF THE INVENTION

The invention relates to therapeutic nucleic acid delivery.

### BACKGROUND OF THE INVENTION

Advances in DNA sequencing technology and prenatal diagnostics, including the ability to detect cell-free fetal DNA in maternal circulation, allow for the diagnosis of many genetic diseases before birth (1, 2). Some of these congenital diseases are currently managed by protein or enzyme replacement therapies after birth and are prime candidates for gene replacement and/or gene editing approaches. Although postnatal therapy is promising for many diseases, the pathology of some diseases begins prior to birth and is irreversible, resulting in prenatal or perinatal death or long-term morbidity (3). Prenatal therapy enables treatment prior to the onset of, or in the early stages of, irreversible pathology to significantly reduce disease morbidity and mortality (4-7). For example, the onset of irreversible disease pathology in some lysosomal conditions begins prior to birth, and the congenital hematologic disease alpha-thalassemia can be associated with hemoglobin Bart's hydrops fetalis resulting in prenatal or early postnatal death (3, 8, 9). Further, glycogen storage diseases and those caused by protein deficiencies, are ideal candidates for prenatal therapy (10-14). Delivering therapeutic nucleic acids or proteins prior to birth has additional advantages based on the normal ontogeny of the fetus. For example, the small fetal size allows for the administration of a maximal therapeutic dose per recipient weight (6). Further, target progenitor cells in multiple organs are more prevalent and highly accessible during gestation, and many physical barriers, such as the blood-brain barrier, are not as developed as they are after birth (7, 15). Lastly, prenatal delivery of nucleic acids may induce immunologic tolerance to the therapeutic protein due to the tolerogenic nature of the fetal immune system (16, 17).

Protein and enzyme replacement therapy could occur via direct protein delivery or nucleic acid delivery (10, 12). Therapeutic protein replacement via mRNA delivery has several potential benefits over delivery of other types of nucleic acids, such as DNA, and whole proteins. For example, unlike DNA, mRNA induces transient protein expression in the cytosol, avoiding the need for nuclear entry without risk of genome integration (18). Unlike direct delivery of proteins, the use of endogenous machinery to produce the therapeutic protein following mRNA delivery allows for natural post-translational modifications to occur (14). However, similar to known delivery barriers in adults, the implementation of mRNA therapeutics for in utero therapy is met with several limitations including mRNA instability leading to rapid degradation and poor cellular uptake due to the negative charge of naked mRNA (19, 20). These limitations preclude the clinical use of naked nucleic acids, including mRNA, in both pre- and post-natal disease management, making it necessary to develop novel mRNA delivery technologies (21, 22).

Common methods for therapeutic nucleic acid delivery include viral- and nonviral-mediated approaches (4, 5, 23, 24). Although viral-mediated delivery of nucleic acids for gene therapy, including prenatal gene therapy (4, 5, 23), holds tremendous promise, nonviral-mediated delivery may be a more suitable alternative (25, 26). Nucleic acid delivery via viral vectors presents the risk of ectopic vector integration, which may lead to persistent transgene expression that may have deleterious consequences for some therapies including gene editing (25, 26). Alternatively, nonviral mRNA delivery approaches can enable transient nucleic acid expression without the risk of genome integration of the carrier vehicle (27). Thus, there is a critical need to develop non-viral and biocompatible nucleic acid delivery technologies to treat prenatal diseases.

The use of nonviral delivery systems has only recently emerged as a technique to enable nucleic acid delivery to fetuses for prenatal therapy (15, 28). Poly(lactic-co-glycolic acid) (PLGA) nanoparticles (NPs) have been shown to induce gene editing in fetal hematopoietic stem cells and mitigate disease in a mouse model of β-thalassemia (15). This important study demonstrated the potential of nonviral approaches for nucleic acid delivery to treat congenital diseases while highlighting the need to develop drug delivery technologies specifically for fetal delivery.

The use of PLGA NPs for nucleic acid delivery is supported by several benefits afforded by the NPs including high biocompatibility and biodegradability. Further, recent studies have advanced techniques of polymeric NP formulation, such as microfluidic devices, have allowed for more precise control over the size of PLGA NPs, overcoming a previous limitation of size control (29-33). Although PLGA and other polymeric systems hold promise for drug delivery to fetuses, the invention disclosed herein relates to developing nonviral, ionizable lipid nanoparticles (LNPs) for this application. LNPs offer small sizes (<100 nm) and yield high cellular uptake, and they have been extensively studied for nucleic acid delivery in adult mice (19, 34, 35). The utilization of ionizable lipids also enables endosomal escape for efficient nucleic acid delivery to the cytosol (36). Further, LNPs offer the ability to design and evaluate new ionizable polyamine-lipid structures within the LNP formulations to optimize platforms for specific applications such as fetal delivery, as disclosed herein.

### SUMMARY OF THE INVENTION

Disclosed herein are ionizable polyamine lipid nanoparticle (LNP) formulations that can utilized as a platform technology for nucleic acid delivery to treat monogenic fetal diseases that do not currently have sufficient therapeutic options in the prenatal setting. These platform formulations yield high hepatic delivery and transfection efficiency with advantageous safety profiles compared to the commercially available, delivery agents such as DLin-MC3-DMA and jetPEI. In addition to the ionizable lipids, these LNPs contain phospholipids, cholesterol, and lipid anchored poly(ethylene) glycol (PEG), which contribute to NP structural integrity, stability, and intracellular mRNA delivery.

LNP formulations encapsulate mRNA to be used in methods for treating congenital disorders. In some methods of the invention, LNP-encapsulated mRNA may be administered to fetuses through the vitelline vein for efficient mRNA delivery to fetal organs. LNPs of the invention enable functional mRNA delivery to fetal livers, lungs and intestines. For example, LNP formulations of the invention may be used to deliver erythropoietin (EPO) mRNA to hepatocytes to elevate EPO protein in the fetal circulation. Accordingly, EPO mRNA LNP formulations of the invention may be used for hepatocyte-mediated protein replacement therapy. we will utilize this platform of novel LNPs to deliver disease-specific, therapeutic nucleic acids to treat congenital disorders.

### BRIEF DESCRIPTION OF THE DRAWINGS

FIG. 1 depicts an overview of LNP formulation and fetal injections for this work. First, novel ionizable core structures were prepared by Michael addition chemistry. Then, the ionizable lipids, PEG-lipid, DOPE phospholipid, and cholesterol were combined into an ethanol phase, and luciferase mRNA was diluted into an aqueous phase. Both phases were combined at controlled flow rates through microfluidic devices. After LNP formulation, LNPs were injected to individual fetuses through the vitelline vein, which directly delivers to sinusoids in the fetal liver. After 4 or 24 hours, fetuses and tissues were extracted for imaging and further analysis.

FIG. 2A depicts chemical structures of the polyamine cores (left) and epoxide terminated alkyl tails (right) that were combined to generate the novel ionizable lipids used in this study. Throughout this paper, LNPs are named for their ionizable lipid component's alkyl tail length (A=C12, B=C14, and C=C16) as well as their polyamine core (numbered 1-5).

FIG. 2B depicts graphed analyses of LNP pKA for representative NPs A-3 and B-4. The pKa for each LNP was calculated by determining the pH that corresponds with normalized TNS fluorescence at 0.5.

FIG. 2C depicts LNP characterization table showing hydrodynamic diameter (intensity), encapsulation efficiency, and pKa for each LNP formulation.

FIG. 3A depicts a schematic (left) and photograph (right) showing the vitelline vein injection in a mouse fetus.

FIG. 3B depicts IVIS imaging showing luciferase expression in a representative dam (left) and within the exposed uterine horn (right). Each fetus was injected via the vitelline vein, extracted, and imaged by IVIS 4 hours after injection. Quantifications are the normalized total flux calculated by dividing the luminescence from the area of interest by the background from each individual image. The normalized total flux was averaged across injected fetuses. * p<0.001 by 1-way ANOVA with posthoc Tukey-Kramer compared to all other treatment groups, unless indicated otherwise, and outliers were detected using Grubbs' test and removed from analysis; minimum n=3/treatment group; error bars represent SEM.

FIG. 3C depicts IVIS images (left) and quantification (right) of luciferase signal in fetuses following surgical removal from dams. Each fetus was injected via the vitelline vein, extracted, and imaged by IVIS 4 hours after injection. Quantifications are the normalized total flux calculated by dividing the luminescence from the area of interest by the background from each individual image. The normalized total flux was averaged across injected fetuses. * p<0.001 by 1-way ANOVA with posthoc Tukey-Kramer compared to all other treatment groups, unless indicated otherwise, and outliers were detected using Grubbs' test and removed from analysis; minimum n=3/treatment group; error bars represent SEM.

FIG. 3D depicts IVIS images (left) and quantification (right) of luciferase signal from livers of fetuses injected with LNPs. Each fetus was injected via the vitelline vein, extracted, and imaged by IVIS 4 hours after injection. Quantifications are the normalized total flux calculated by dividing the luminescence from the area of interest by the background from each individual image. The normalized total flux was averaged across injected fetuses. * p<0.001 by 1-way ANOVA with posthoc Tukey-Kramer compared to all other treatment groups, unless indicated otherwise, and outliers were detected using Grubbs' test and removed from analysis; minimum n=3/treatment group; error bars represent SEM.

FIG. 4A depicts IVIS images showing LNP-mediated mRNA delivery to fetal lungs (left) and quantification (right) of luciferase signal in the lungs. Normalized total flux was averaged across injected fetuses. * p<0.05 by one-way ANOVA with posthoc Tukey-Kramer compared to all other treatment groups, unless indicated otherwise; minimum n=3 fetuses/treatment group; error bars represent SEM.

FIG. 4B depicts IVIS images showing LNP-mediated mRNA delivery to fetal intestines (left) and quantification (right) of luciferase signal in the intestines. Normalized total flux was averaged across injected fetuses. * p<0.05 by one-way ANOVA with posthoc Tukey-Kramer compared to all other treatment groups, unless indicated otherwise; minimum n=3 fetuses/treatment group; error bars represent SEM.

FIG. 5A shows GFP expression in fetal livers 24 hours after injection with LNPs A-3.luc or B-4.luc with encapsulated GFP mRNA, free mRNA, or PBS, showing that LNPs can deliver multiple mRNAs. All tissue sections were imaged with a 400 ms exposure time.

FIG. 5B shows EPO content in fetal livers at 4 hours (left) or 24 hours (right) post-injection of LNPs pA-3.luc or pB-4.luc with encapsulated EPO mRNA, or PBS. EPO concentrations were averaged across three fetuses per treatment group and analyzed by two-way ANOVA comparing mean EPO concentration amongst treatment groups; * p<0.02, ** p<0.001; error bars represent SEM.

FIG. 6A depicts the percent survival of fetuses injected with LNPs at E16 and surgically delivered at E19. Survival was determined immediately following extraction. Error bars represent standard deviation from three dams following injection to every fetus in each dam.

FIG. 6B depicts liver enzyme analysis from fetal liver tissue collected at E19 following injection at E16. Measured AST or ALT units were normalized by dividing by protein concentration from fetal liver tissue. The following cytokines were out of range of the instrument and is therefore not shown: IFNγ, IL-10, IL-12 (p40), IL-12 (p70), IL-1b, IL-2, IL-4, TNFα, VEGF. n=3 dams/treatment group. * p<0.02 and ** p<0.0001 by 2-way ANOVA compared to each treatment group for each cytokine; n=3 dams/treatment group. Error bars represent SEM with outliers detected by Grubbs' test and removed from analysis.

FIG. 6C depicts cytokine analysis from fetal livers collected immediately following surgical delivery at E19. The following cytokines were out of range of the instrument and is therefore not shown: IFNγ, IL-10, IL-12 (p40), IL-12 (p70), IL-1b, IL-2, IL-4, TNFα, VEGF. n=3 dams/treatment group. * p<0.02 and ** p<0.0001 by 2-way ANOVA compared to each treatment group for each cytokine; n=5 dams/treatment group. Error bars represent SEM with outliers detected by Grubbs' test and removed from analysis.

FIG. 6D depicts liver enzyme analysis, based on AST and ALT plasma levels in fetuses injected with LNPs at E16 and surgically delivered at E19.

FIG. 6E depicts complement system activation, based on C3 and C4 plasma levels fetuses injected with LNPs at E16 and surgically delivered at E19.

FIG. 6F depicts cytokine analysis from plasma collected from dams at E19 prior to surgical delivery of the injected fetuses. The following cytokines were out of range of the instrument and is therefore not shown: IFNγ, IL-10, IL-12 (p40), IL-12 (p70), IL-1b, IL-2, IL-4, TNFα, VEGF. n=3 dams/treatment group. * p<0.02 and ** p<0.0001 by 2-way ANOVA compared to each treatment group for each cytokine; n=3 dams/treatment group. Error bars in (B-F) represent SEM with outliers detected by Grubbs' test and removed from analysis.

FIG. 7 depicts chemical structures of the excipients used in the LNP formulations, including C14-PEG2000 (PEG-lipid conjugate), DOPE (phospholipid), and cholesterol.

FIG. 8 depicts IVIS images showing luciferase expression in intestine, lung, kidney, heart, and brain from fetuses treated with each LNP formulation.

FIG. 9 depicts examples of the regions of interest (ROI) used to calculate normalized luminescence. For all images, an ROI (ROI 1) was placed over the fetus or tissue of interest and a second ROI of the same size was placed over an area of the background. To calculate the normalized luminescence, the total flux from ROI 1 was divided by the total background flux to account for variability in background total flux between samples and experiments. The same quantification technique was used for all experiments, and the same size ROIs were used for all samples for each tissue. It is important to note that the images shown in this supplemental figure are not at the same luminescence scale and are only meant to demonstrate the ROIs used for quantification of each tissue.

FIG. 10A depicts LC-MS spectra of the polyamine-lipid core used to prepare A-3 LNPs.

FIG. 10B depicts LC-MS spectra of the polyamine-lipid core used to prepare B-4 LNPs.

FIG. 11A depicts a comparison of the delivery efficiency for crude and purified polyamine-lipid cores that were used to prepare LNPs A-3.luc and B-4.luc in fetuses. Images were taken 4 hours or 24 hours post-injection of LNPs. n=3-7 fetuses/treatment group; error bars represent SEM.

FIG. 11B depicts a comparison of the delivery efficiency for crude and purified polyamine-lipid cores that were used to prepare LNPs A-3.luc and B-4.luc in fetal livers. Images were taken 4 hours or 24 hours post-injection of LNPs. n=3-7 fetuses/treatment group; error bars represent SEM.

FIG. 12A depicts the percent survival of fetuses injected at E16 and surgically delivered at E19. Survival was determined immediately following surgical delivery of fetuses.

FIG. 12B depicts cytokine analysis from fetal livers immediately following surgical delivery at E19. n=3 fetuses/treatment group and outliers were detected using Grubbs' test; error bars represent SEM.

FIG. 13 depicts an assessment of mRNA immunogenicity without LNP encapsulation. Monocyte-derived human dendritic cells were transfected with luciferase or EPO mRNA and IFN-α levels in the culture media was evaluated after 24 hours. Error bars represent SEM.

FIG. 14 depicts thiol-maleimide chemistry to attach peptides and/or antibodies to the PEG component of the LNP such that the LNP can be targeted to the intended organ.

FIG. 15 illustrates that modification of LNP with a transferring receptor targeting peptide results in ˜2-3-fold increase in brain targeting.

### DETAILED DESCRIPTION OF THE INVENTION

The invention described here relates to methods and compositions for the delivery of prenatal therapeutics, enzyme replacement therapy, or gene therapy to a fetus in need thereof, comprising introducing ionizable lipid nanoparticles (LNPs) nanoparticles comprising a therapeutic mRNA composition into the circulation of the fetus in need of treatment, wherein the ionizable LNPs deliver the therapeutic mRNA composition.

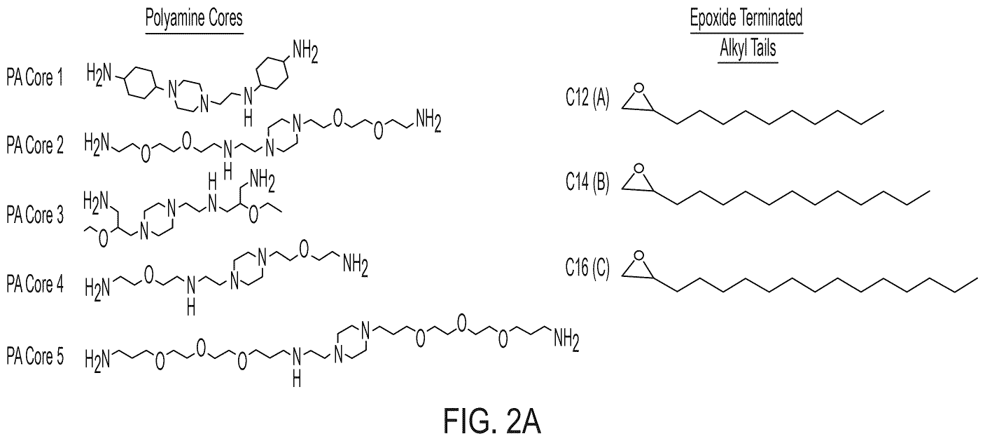

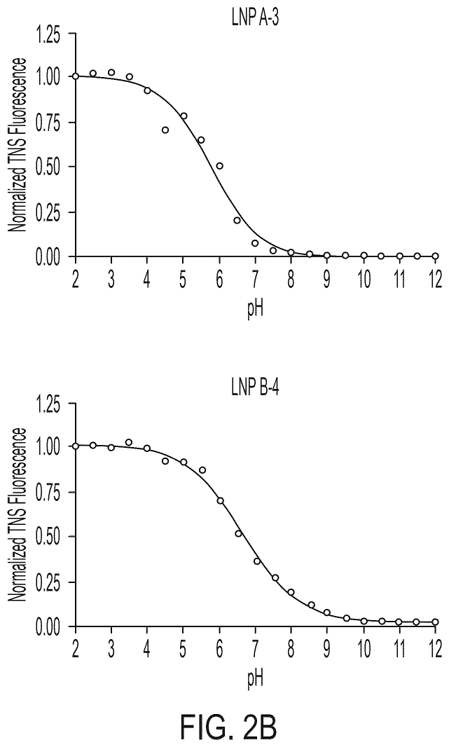

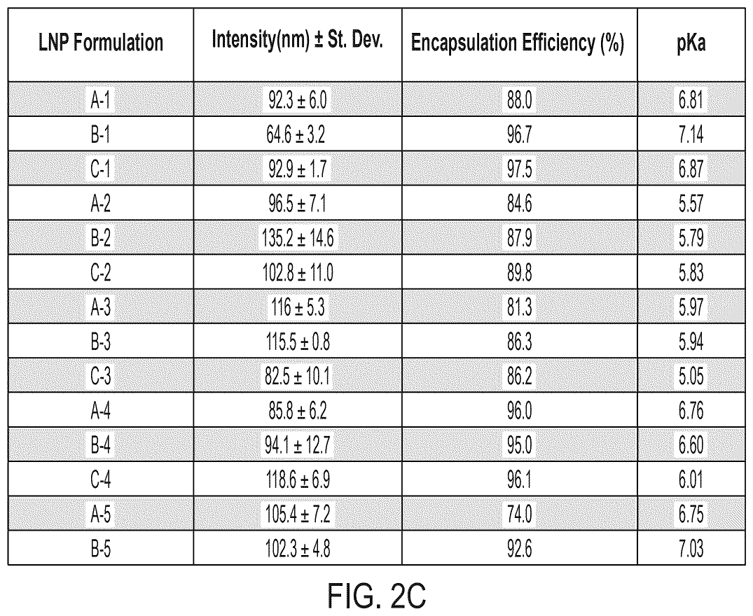

Generally, ionizable LNP formulations of the invention contain one or more ionizable polyamine-lipids, cholesterol, 1,2-Dioleoyl-sn-glycero-3-phosphoethanolamine (DOPE), and a pegylated lipid (PEG-lipid). In some formulations of the invention, the formulation contains one or more polyamine lipid selected from the polyamines depicted from the chemical structures in FIGS. 2A and 2C. The PEG-lipid of an ionizable LNP formulation of the invention may be, but is not limited to 1,2-dimyristoyl-snglycero-3-phosphoethanolamine-N-[methoxy (polyethylene glycol)-2000 (C14-PEG2000). For example, an ionizable LNP formulation of the invention may contain one or more ionizable polyamine lipids, cholesterol, DOPE, and C14-PEG2000 are at respective molar ratios of about 35:46.5:16:2.5

The polyamine head group of an ionizable LNP particle of the invention may incorporate, but are not limited to, any one or more of the epoxide-terminated alkyl tails disclosed in FIG. 2A. Examples of ionizable polyamine lipids of the invention with incorporated epoxide-terminated alkyl tails include compounds A-1 through A-5, B-1 through B-5, and C-1 through C-4 as described in FIGS. 2A and 2C.

Ionizable LNPs of the invention are nanoparticles and may range in size from 60 nm to 140 nm. In some ionizable LNP formulations of the invention, the mean size of the LNPs may range from about 64-136 nm.

Ionizable LNPs of the invention may also be modified by incorporating a delivery target-specific antibody-conjugated PEG, or a peptide-conjugated PEG. For example, an ionizable LNP formulation of the invention may be modified to target hematopoietic stem cells (HSCs) or progenitor cells, brain, heart, or lung cells.

The pharmacokinetics of ionizable LNPs of the invention sufficient to allow fusion of i.v.-administered LNPs with target endosomal membranes to promote release of encapsulated mRNA into the cytosol. Accordingly, i.v.-administered LNPs of the invention have a pKa of less than 7. For example, the pKa of an ionizable LNP formulation of the invention may be about 5.5 to about 7.2.

As indicated above, ionizable LNP formulations of the invention may be introduced into the circulation of a fetus intravenously to deliver therapeutic mRNA to an organ or cellular target. In certain ionizable LNP formulations of the invention, the formulation may additionally include one or more of prostaglandin e2, diprotin A, and IL-37 to improve survival and proliferation of HSCs.

As stated above, ionizable LNP formulations of the invention may be administered to deliver therapeutic mRNA to target fetal tissues. Therefore, methods of the invention are useful for delivering mRNA-based prenatal therapeutics and enzyme replacement therapies.

### EXAMPLES

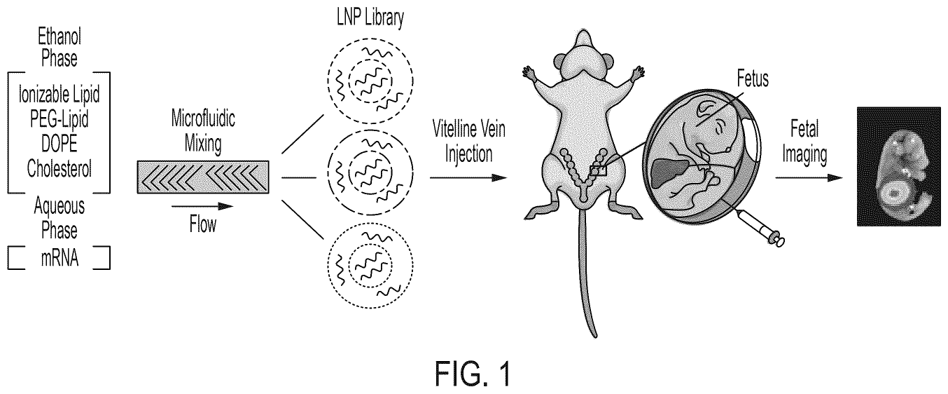

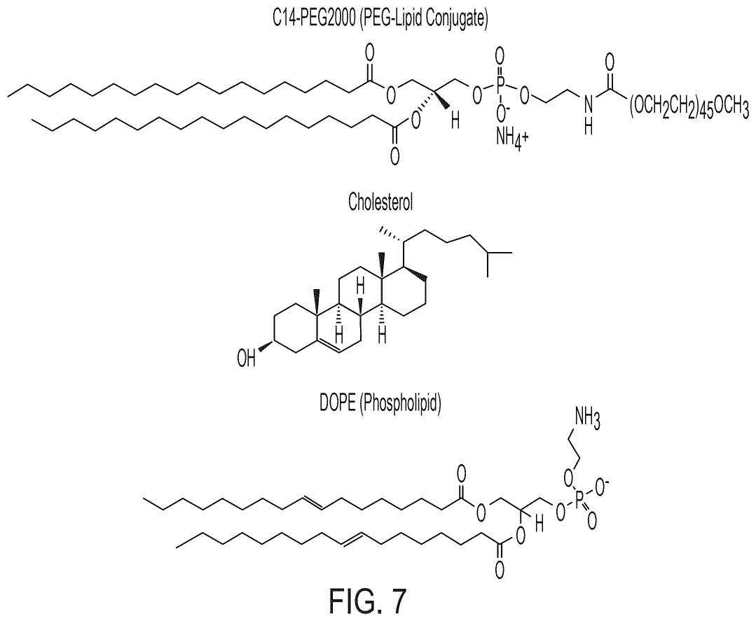

Example 1: Characterization of the LNP Library. A library of 14 LNPs was prepared as previously described by first synthesizing ionizable lipids using Michael addition chemistry (FIGS. 1, 2A) (34). During this process, the polyamine molecules react with the alkyl tails to form the polyamine-lipid cores. The naming convention of LNP formulations throughout this paper reflects both the alkyl tail length (A=C12, B=C14, and C=C16) as well as the unique polyamine core (labelled numerically 1-5) of the ionizable lipid component. For example, LNP A-3 is comprised of the C12 epoxide-terminated alkyl tail reacted with the polyamine core labeled “3” in FIG. 2A. These ionizable lipids were then mixed with cholesterol, 1,2-Dioleoyl-sn-glycero-3-phosphoethanolamine (DOPE) phospholipid, PEG-lipid conjugates, and mRNA via perfusion through microfluidic devices that are designed with herringbone features to induce chaotic mixing (FIGS. 1, 2A, 7) (39). In LNPs, the ionizable lipid enables cellular uptake and endosomal escape such that the encapsulated mRNA is delivered to the cytosol (40). The excipients (DOPE, cholesterol, and PEG-lipids) were chosen based on previous studies demonstrating that their inclusion yields optimal mRNA delivery in adult mice (35). Specifically, DOPE and cholesterol provide stability to the lipid bilayer, reduce mRNA leakage, and may assist in endosomal escape (37, 38). The PEG-lipids enhance overall LNP stability and extend circulation (35).

LNP formulations were characterized by size, pKa, and mRNA encapsulation efficiency (FIG. 2B, C). The hydrodynamic diameter (by intensity measurements using dynamic light scattering) for all LNP formulations ranged from 64.6-135.2 nm (FIG. 2C). Only one LNP formulation had a polydispersity (PDI) value above 0.3, with all others having a PDI value less than 0.3 indicating monodisperse LNPs. Each LNP formulation was evaluated for its ability to encapsulate mRNA using Ribogreen® assays, and all encapsulation efficiencies were high, ranging from 74%-97.5% (FIG. 2C). Lastly, LNPs were assessed for their pKa, the pH at which the LNPs are 50% protonated. This reflects how pH affects their ability to escape acidic endosomal compartments inside cells (FIG. 2B, C). pKa values <7.0 indicate that the LNPs will become protonated in endosomes causing the lipids to fuse with the endosomal membrane for release of mRNA into the cytosol, and pKa values of 6-7 are most commonly reported for in vivo nucleic acid delivery (36, 41, 42). The measured pKa values from our LNP library ranged from 5.57-7.14, indicating that many of the LNPs were within the desired range for nucleic acid delivery.

Polyamine-Lipid Synthesis and Purification. The ionizable lipid cores were prepared via Michael addition chemistry as previously described (34). Briefly, the polyamine cores (purchased from Enamine, Inc., Monmouth Jct., NJ) were combined with excess moles of lipid epoxide needed to saturate the amines in 4 mL amber vials with a magnetic stir bar. The lipid epoxides used in this study were epoxydodecane (C12), epoxytetradecane (C14), or epoxyhexadecane (C16) (Sigma-Aldrich, St. Louis, MO). The vial was sealed, and the reaction was mixed for 2 days at 80° C. The crude reaction mixture was dried using a Rotovap R-300 (Buchi, New Castle, DE), and the crude reactions were used for screening the library with luciferase mRNA. The A-3 and B-4 reaction mixtures were further characterized by liquid chromatography-mass spectrometry (LC-MS). The resultant fractions from the reaction were separated using a CombiFlash NextGen 300+ (Teledyne ISCO, Lincoln, NE) against a gradient of 100% methanol to 100% of a solution comprised of 75% dichloromethane, 22% methanol, and 3% ammonium hydroxide over 55 min. Each peak was collected and dried, and the molecular weight of the fully saturated product was confirmed by LC-MS. This purified product was used to deliver human erythropoietin (EPO) mRNA.

Formulation of Lipid Nanoparticles. The ionizable polyamine-lipid cores, prepared as described above, or DLin-MC3-DMA (MedChem Express, Monmouth Junction, NJ), were combined into an ethanol phase with cholesterol (Sigma-Aldrich), 1,2-dioleoyl-sn-glycero-3-phosphoethanolamine (DOPE, Avanti, Alabaster, AL), and 1,2-dimyristoyl-snglycero-3-phosphoethanolamine-N-[methoxy (polyethylene glycol)-(ammonium salt) (C14-PEG2000, Avanti) at molar ratios of 35:46.5:16:2.5, respectively, in a total volume of 112.5 μL. A separate aqueous phase was prepared consisting of 25 μg luciferase (TriLink Biotechnologies, San Diego, CA) or EPO (TriLink Biotechnologies) mRNA and 10 mM citrate buffer (pH 3) in a total volume of 337.5 μL. All mRNA was N1-methyl-pseudo-U capped with CleanCap technology offered by TriLink Biotechnologies. The ethanol and aqueous phases were combined through channels in a microfluidic device using a syringe pump as previously described (39). NPs were dialyzed against PBS for 2 hours prior to sterile filtration through syringe filters with 0.2 μm pores and stored at 4° C. JetPEI (Polyplus Transfection, New York, NY)-mRNA complexes were prepared according to manufacturer protocols with N/P=7. All materials were prepared and handled RNase-free throughout the synthesis, formulation, and characterization steps.

Nanoparticle Characterization. For dynamic light scattering (DLS) and zeta potential measurements, 10 μL of each NP solution was combined with 1 mL 1×PBS in 4 mL disposable cuvettes (for DLS) or zeta cuvettes (for zeta potential). Samples were run on a Zetasizer Nano (Malvern Instruments, Malvern, UK), and the reported measurements are averages+/−standard deviation from three runs. Surface ionization measurements to calculate the pKa of each NP formulation were conducted as previously described (36). Aliquots of a buffered solution containing 150 mM sodium chloride, 20 mM sodium phosphate, 20 mM ammonium acetate, and 25 mM ammonium citrate were each adjusted to pH 2-12 in 0.5 increments. 200 μL of each pH-adjusted solution was combined with 5 μL of each NP formulation in black 96-well plates in triplicate. 6-(p-toluidinyl) naphthalene-2-sulphonic acid (TNS) was added to each well for a final TNS concentration of 6 μM and the fluorescence intensity was read on an Infinite 200 Pro plate reader (Tecan, Morrisville, NC) (ex 322 nm/em 431 nm). The fluorescence intensity versus pH was plotted and the pKa was calculated to be the pH that corresponded to 50% protonation.

Encapsulation efficiencies were calculated using Quant-iT Ribogreen (ThermoFisher, Waltham, MA) assays as previously described (70). Two microcentrifuge tubes of 350 μL of each NP solution was aliquoted, and 1% vol/vol Triton X-100 (Sigma) was added to one of the tubes. After 10 min, NPs (with and without Triton X-100) and RNA standards were plated in triplicate in black 96 well-plates and the fluorescent Ribogreen reagent was added per manufacturer instructions. Fluorescence intensity was read on the plate reader (ex 490 nm/em 520 nm). To quantify encapsulation efficiency, background signal was subtracted from each well and triplicate wells were averaged. RNA content was quantified by comparing samples to the standard curve. Encapsulation efficiency was calculated according to the equation

$$\frac{B - A}{B} \times 100 ,$$

where A is the RNA content before treatment with Triton X and B is the RNA content from samples treated with TritonX.

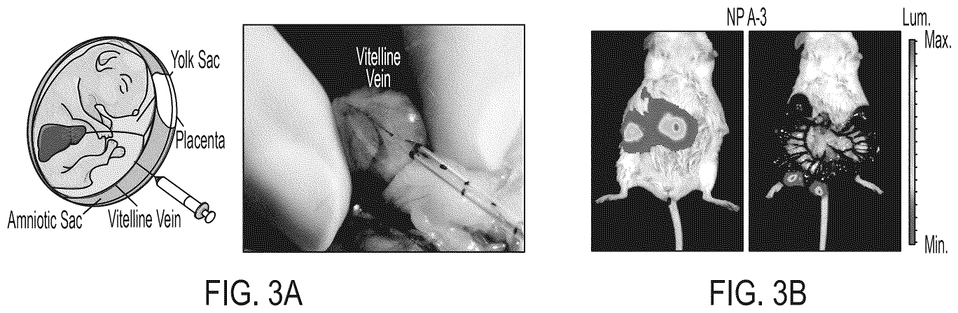

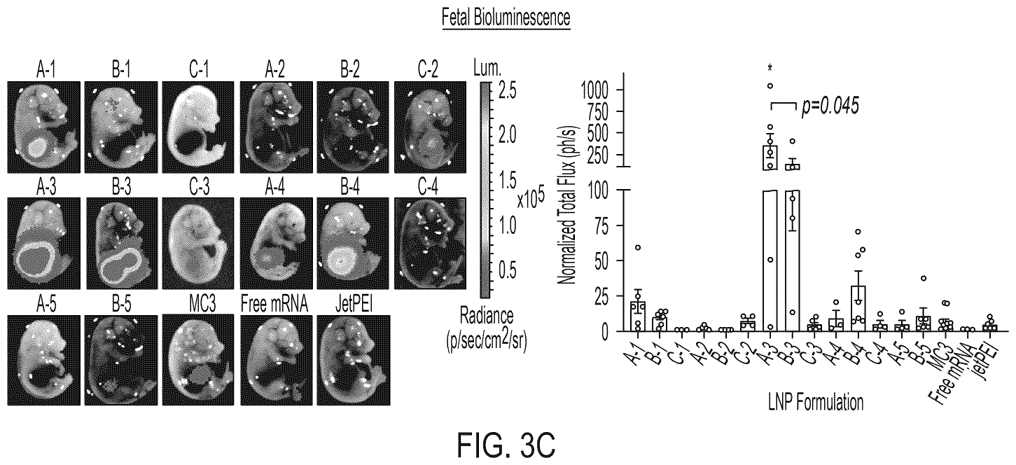

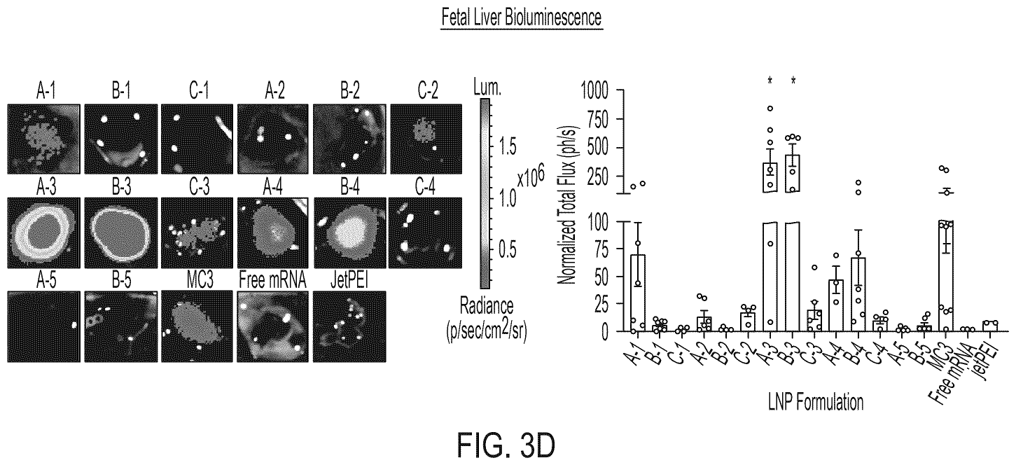

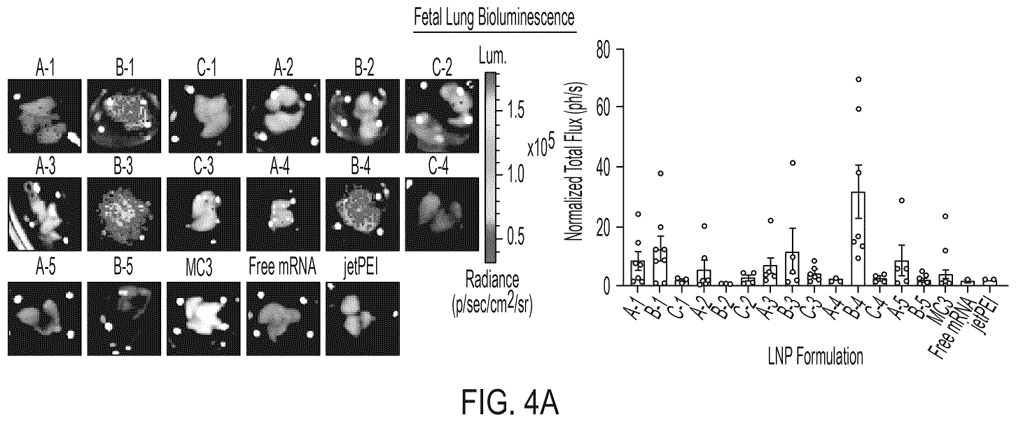

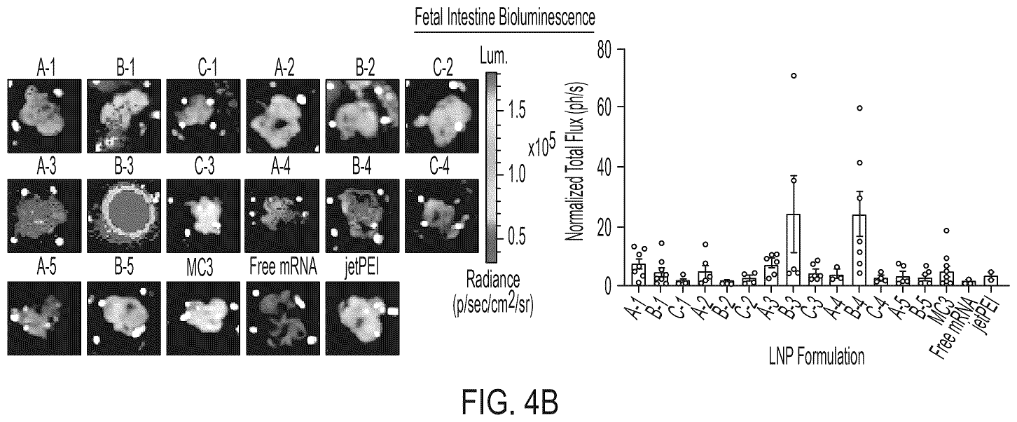

Example 2: In Utero mRNA Delivery. Prenatal mRNA delivery via our LNPs using luciferase mRNA were evaluated, as this system enables direct visualization of transfection efficiency using an in vivo imaging system (IVIS). Thus, imaged luminescence indicates both LNP delivery as well as mRNA functionality. LNPs encapsulating luciferase mRNA (LNP.luc) were injected into gestational day (E) 16 mouse fetuses (minimum n=3 fetuses/LNP formulation depending on the number of fetuses in each dam) via the vitelline vein, and injected dams and fetuses were assessed by IVIS 4 hours after injection FIGS. 1, 3A). The vitelline vein drains directly into the fetal portal circulation and thus this model represents a mid-gestation umbilical vein injection in a human fetus. Each mouse fetus has its own gestational sac and vitelline vein such that the vitelline vein injectate of one fetus does not cross over to additional fetuses (43). The sample size for each treatment varies because there is a range in the number of fetuses within the uterine horn of each dam. Thus, half of the fetuses in each dam were injected with a LNP formulation while the other half were injected with phosphate buffered saline (PBS) as an internal negative control for imaging. All the data shown in FIGS. 3-4 represent only fetuses injected with LNPs and not the negative PBS-injected controls.

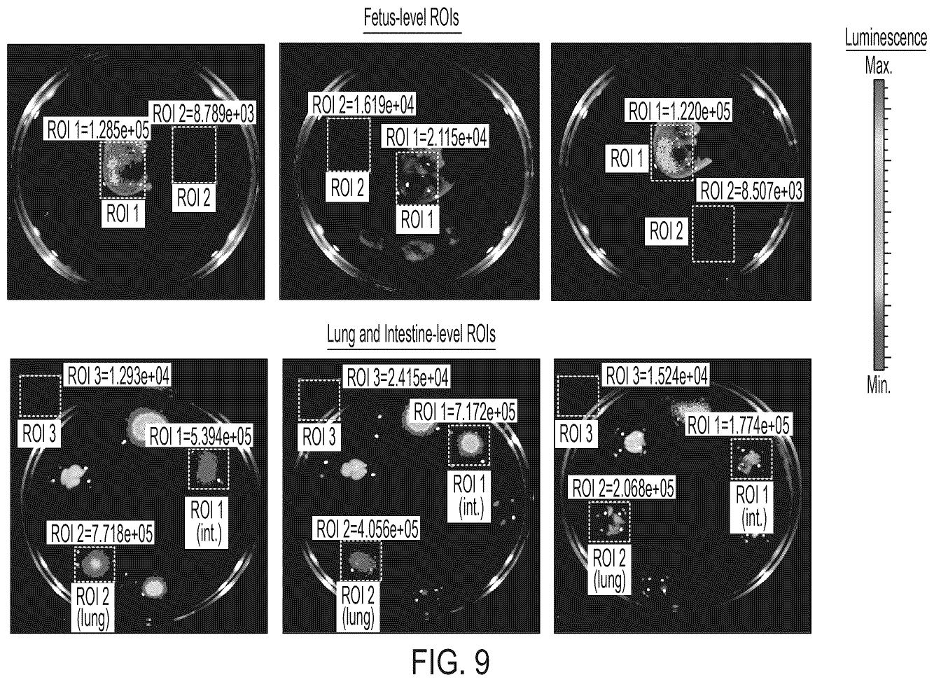

Analysis of pregnant dams following fetal injection revealed strong luciferase signal localized to the fetus for several LNP formulations and no signal in any fetuses injected with PBS (FIG. 3B). Additionally, no maternal tissues had luciferase signal, suggesting an absence of transplacental migration of the LNPs from fetus to dam or other fetuses in the uterine horn. After imaging the dams, experimental and control fetuses were surgically removed to allow for more precise comparison of the efficiency of mRNA delivery between different LNPs (FIG. 3C, 3D, 8). The fetuses were imaged individually by IVIS (FIG. 3C), and the normalized luminescent signal was subsequently quantified (FIG. 3D). For each image, a rectangular region of interest (ROI) was placed over the fetus, and a second ROI of the same size was placed over an area of the image not of the fetus as background. The same sized ROIs were used in every image for each quantified tissue, and ROIs were consistently placed over the same area of each fetus or tissue. (FIG. 9). The reported normalized luminescence represents the total flux from the ROI placed over the fetus divided by the total flux from background ROI. By quantifying the luminescent data in this manner, the minor differences in background luminescence between fetuses treated on different days were accounted for. Some LNP formulations yielded greater mRNA delivery compared to others, indicating that the ionizable lipid within the LNPs strongly dictates their ability to deliver mRNA to fetuses. Specifically, LNPs A-1, A-3, B-3, and B-4, yielded the strongest luciferase signal (FIG. 3C, 3D, 8), and no fetuses injected with free mRNA yielded detectable luciferase signal at the imaging parameters used in these experiments, indicating the need for LNPs for efficient mRNA delivery.

Animal Experiments. All animal use was in accordance with the guidelines and approvals from the Children's Hospital of Philadelphia's (CHOP) Institution of Animal Care and Use Committee (IACUC). Balb/c mice were mated in our breeding colony (originally purchased from Jackson Laboratories, Bar Harbor, ME) and maintained in the Laboratory Animal Facility of the Colket Translational Research Building at CHOP. Females of breeding age were paired with males and separated at 24 hours in order to achieve time-dated pregnant dams.

In vivo Studies. Fetuses of time-dated pregnant Balb/c dams were injected at gestational day 16 (E16) as previously described (71). Briefly, under isoflurane anesthesia, a midline laparotomy was performed to expose the uterine horns. A dissecting microscope was used to identify the vitelline vein of each fetus and a total volume of 5 μL (38.4 ng/ML mRNA) of the NP formulation was injected using an 80 μm beveled glass micropipette and an automated microinjector (Narishige IM-400 Electric Microinjector, Narishige International USA, Inc., Amityville, NY). After successful injection, confirmed by visualizing clearance of the blood in the vein by the injectate, the uterus was returned to the peritoneal cavity and the abdomen closed with a single layer of absorbable 4-0 polyglactin 910 suture.

Luciferase Imaging. Mice were imaged either 4 or 24 hours after injections with NPs. Luciferase imaging was conducted on an in vivo imaging system (IVIS, PerkinElmer, Waltham, MA). 10 minutes prior to imaging, dams were injected intraperitoneally with 150 mg/kg D-Luciferin, potassium salt (Biotium, Fremont, CA). Anesthetized mice were then placed supine into the IVIS, and luminescence signal was detected with a 60 second exposure time. Next, a midline laparotomy was performed to expose the uterine horns, and luciferase imaging was repeated. Dams were then sacrificed, and the fetuses were surgically extracted and placed in 1× phosphate buffered saline (PBS) on ice. Fetuses were individually imaged by IVIS with 60 second exposure times. After imaging, the fetuses were dissected, and the liver, intestines, kidneys, heart, lungs, and brain were imaged by IVIS. Image analysis was conducted in the LivingImage software (PerkinElmer). To quantify luminescent flux, a rectangular region of interest (ROI) was placed over each fetus or organ of interest, and an ROI of the same size was placed in an area without any luminescent signal in the same image. Normalized flux was calculated by dividing the total flux from the ROI over the fetus by the total flux from the background ROI. The representative images shown represent those images with quantified normalized luminescence in close proximity to the average value calculated for each LNP formulation. For GFP imaging experiments, individual fetal organs were dissected and imaged using a fluorescent stereoscopic microscope (MZ716FA, Leica, Heerburg, Switzerland).

Example 3: Comparison of the Invention to State-of-the-Art Polymeric Delivery Systems. LNPs disclosed herein which outperform benchmark, state-of-the-art lipid and polymeric delivery systems DLin-MC3-DMA and jetPEI. LNP platforms disclosed herein were compared against the widely studied in vivo nucleic acid delivery systems, DLin-MC3-DMA (MC3) and jetPEI, for in utero delivery (44-47). Importantly, MC3 and jetPEI have been evaluated for nucleic acid delivery in clinical trials, making their comparison to our LNPs a critical benchmark for in vivo mRNA delivery (45, 48, 49). Further, the MC3 lipid was recently approved by the U.S. Food and Drug Administration for clinical use for siRNA delivery to treat polyneuropathy in patients with hereditary transthyretin-mediated amyloidosis (45-47). Fetuses injected with jetPEI (jetPEI.luc) complexes yielded only a modest increase in luminescence compared to free luciferase mRNA 4 hours post-injection (FIG. 3C, 8). Many of our LNP formulations were more efficient for mRNA delivery compared to jetPEI.luc, and our top-performing LNP A-3.luc, demonstrated a 75-fold increase in luminescence over jetPEI.luc. As a second commercially available comparison, the ionizable lipid MC3 into LNPs (MC3.luc) was incorporated to directly compare in utero mRNA delivery between our ionizable lipids and this gold standard lipid. MC3.luc delivered mRNA to fetal livers, although not to the same extent as our top performing LNPs A-3.luc and B-4.luc. Quantification of normalized total flux in the fetal livers revealed a 3.5-fold and 4-fold decrease in luminescence compared to LNPs A-3 or B-3, respectively. Collectively, these results indicate that LNPs developed in this study induced greater in utero protein expression in fetal livers than benchmark lipid and polymeric delivery systems currently utilized for nucleic acid delivery.

### Example 4: Intravascular LNP Injection Results in mRNA Delivery Primarily to Fetal Livers

Imaging of the pregnant dams and individual fetuses demonstrated the ability of LNP.luc formulations to selectively deliver mRNA to the fetus while avoiding crossover to the dam. Which, if any, organs experienced preferential accumulation of any of the LNP formulations was also evaluated. The liver, lungs, brain, kidney, heart, and intestines from injected fetuses were isolated and analyzed by IVIS at 4 hours post treatment. The brightest signal was detected in livers from fetuses injected with LNPs (FIG. 3D, 8). This is likely due to a first-pass effect, as the vitelline vein drains directly into the portal circulation and forms the hepatic sinusoids (43). The normalized total luminescent flux from fetal livers injected with the various LNP formulations correlated well with the normalized total luminescent flux from the whole fetus (FIG. 3C compared to FIG. 3D) indicating most of the signal noted on whole fetal analyses resulted from LNP-mediated mRNA delivery to the fetal liver.

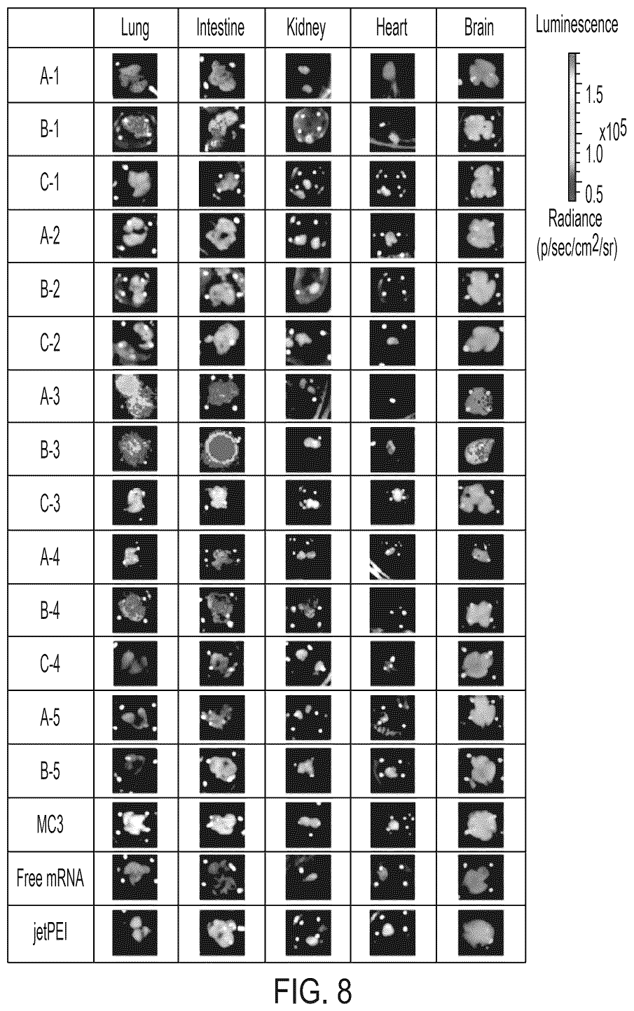

Analyses of kidneys and hearts following delivery of any of the LNP.luc formulations demonstrated minimal detected luminescent signal at the scale range used in these studies (FIG. 8). A low level of luminescent signal was noted in the brains of fetuses injected with a few LNPs, such as B-3.luc (FIG. 8). In addition to demonstrating high levels of liver bioluminescence, fetuses injected with LNPs A-3.luc, B-3.luc and B-4.luc had luminescent signal in the lungs and intestines (FIGS. 4A, 4B, 8). Since both LNPs were comprised of the C12 or C14 epoxide-terminated alkyl tails, it is anticipated that the ability of these LNPs to deliver mRNA to the lung and intestines is due to the combination of similar polyamine core structures (polyamine core 3 is a branched form of polyamine core 4) in each of these formulations with these alkyl tails. However, further testing is needed to evaluate the specific mechanism by which these formulations can surpass the liver to reach the lungs and intestines. Finally, LNP MC3.luc delivered mRNA to the liver, with minimal delivery to lungs and intestines, and luciferase expression induced by jetPEI.luc was not observed in any organs at the imaging scales used here (FIGS. 3D, 8).

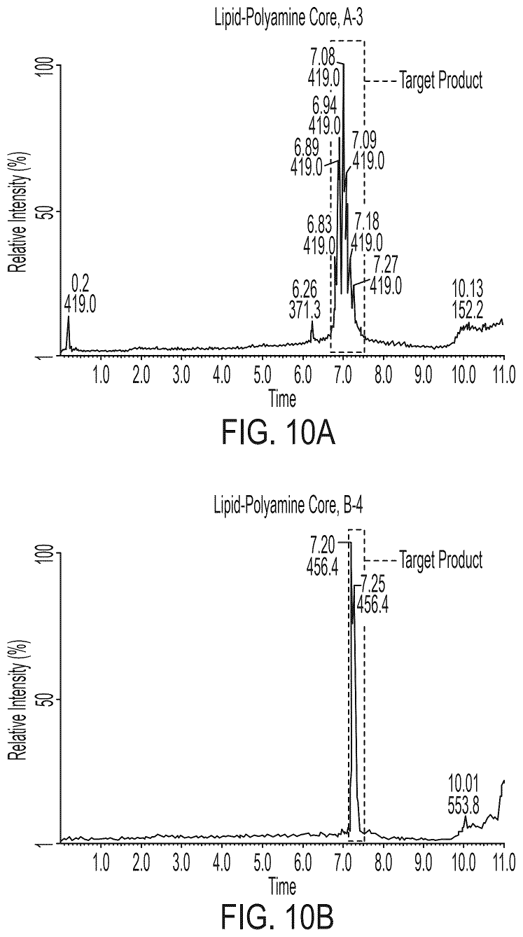

Example 5: Fully Saturated lonizable Lipids Induce Maximal mRNA Delivery. Since the polyamine-lipid synthesis contained more than one level of alkyl chain substitutions (FIG. 9), the mRNA delivery capabilities of only the fully saturated core was evaluated. To do this, the fully saturated polyamine-lipid core by flash chromatography and used the purified material, rather than the crude material, was purified to prepare the A-3 and B-4 LNP formulations (termed pA-3.luc and pB-4.luc, respectively). The purified products were confirmed to be fully saturated with the lipid epoxides by liquid chromatography-mass spectrometry (LC-MS) (FIG. 10).

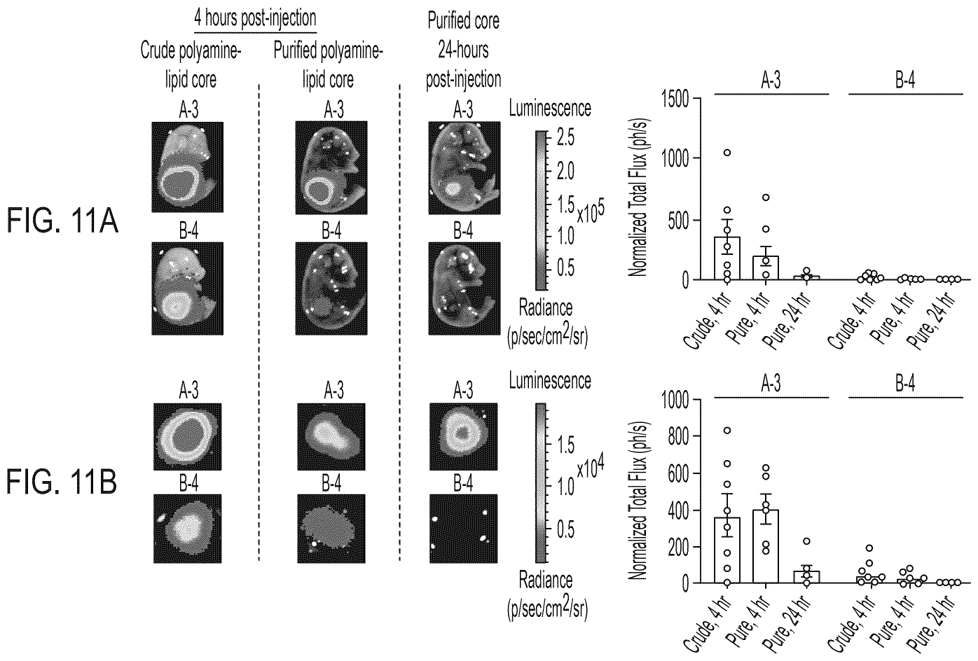

The efficiency of luciferase mRNA delivery by LNPs pA-3.luc and pB-4.luc were compared to that achieved via delivery by LNPs A-3.luc and B-4.luc (prepared with crude material) to ensure that the fully saturated ionizable lipids were primarily responsible for mRNA delivery. Injected fetuses were imaged by IVIS after 4- and 24-hours post-injection (FIG. 11). There was only a modest reduction in luciferase signal in the whole fetus as well as isolated livers following injection with the purified LNPs. These findings are consistent with previous studies and believed to be due to the polyamine cores in the crude mixture having a broad range of alkyl chain substitutions yielding higher mRNA delivery (50). However, given their clinical relevance and only a modest difference in mRNA delivery, LNPs pA-3 and pB-4 were utilized in the subsequent studies to induce hepatic protein expression. As expected, the luciferase signal was transient; the brightest signal was detected at 4 hours post-injection with decreasing levels at 24 hours post injection. Moving forward, there is potential to study repeated doses of LNPs, additional modifications to the mRNA cargo, or more permanent gene therapies, for prolonged therapeutic efficacy (FIG. 11).

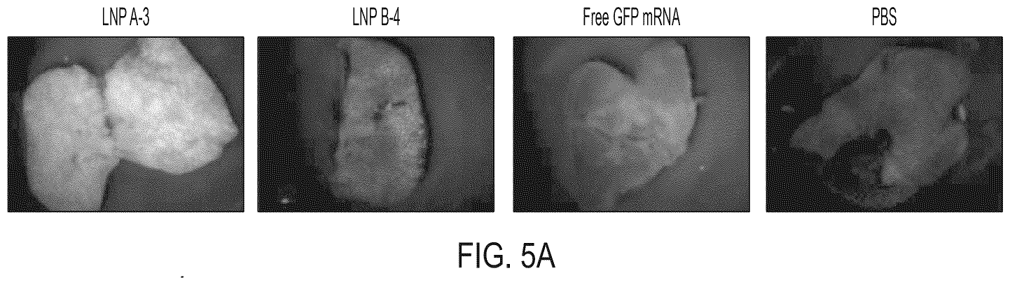

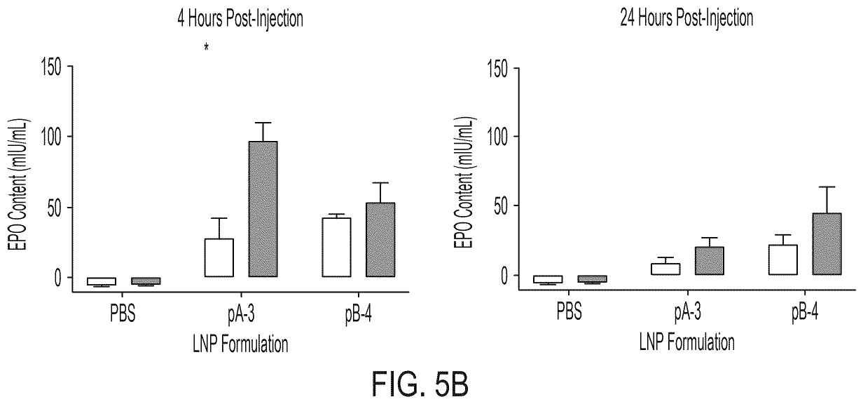

Example 6: LNPs Enable Delivery of GFP mRNA and Erythropoietin mRNA as a Model for Protein Replacement Therapy. To demonstrate that LNPs are robust and can deliver multiple types of mRNAs to fetuses, GFP mRNA, which is detectable by fluorescence analysis techniques, was encapsulated within LNPs A-3.luc and B-4.luc and delivered via the vitelline vein to E16 fetuses, and GFP expression was assessed 24 hours post-injection via fluorescent stereomicroscopy and flow cytometry. Similar to luciferase expression following injection of LNPs A-3.luc and B-4.luc, GFP expression was predominantly in the fetal livers (FIG. 5A) suggesting that the results of the LNP.luc screen are translatable to the prenatal delivery of other mRNAs. Further, treatment with LNP A-3.luc resulted in stronger GFP fluorescence in the liver compared to treatment with LNP B-4.luc, free GFP mRNA, or PBS as demonstrated by both imaging and flow cytometry (FIG. 5A, B). For flow cytometry, fetal livers were processed into a single cell suspension and stained for CD45, which is a marker for hematopoietic cells. The CD45-population was used for analysis to determine the population of GFP-expressing cells from the liver tissue, which revealed that LNPs A-3 and B-4 yielded 2.8% and 0.7% GFP+ cells, respectively. Comparatively, fetuses treated with PBS resulted in 0% of GFP+ cells, demonstrating that any GFP+ cells following treatment with our LNPs is a result of successful mRNA delivery. These results agree with our luciferase data suggesting that LNP A-3 yields higher mRNA delivery to fetal livers compared to LNP B-4 or PBS.

Lastly, to demonstrate the therapeutic potential of LNPs, prenatal delivery of human erythropoietin (EPO) mRNA in LNP pA-3 (LNP pA-3.EPO) and LNP pB-4 (LNP pB-4.EPO) was assessed. Successful liver delivery with LNPs pA-3.EPO or pB-4.EPO would result in hepatic production of EPO protein which is then secreted into the circulation. This model is relevant to a number of enzyme deficiency disorders, such as the lysosomal storage diseases, which cause irreversible damage prior to birth and for which hepatic production and secretion of the deficient enzyme is being pursued as a viable therapy (3). LNP pA-3.EPO and pB-4.EPO were injected through the vitelline vein into E16 fetuses at two different doses (5 mL=190 ng EPO mRNA or 20 mL=760 ng EPO mRNA) and fetal livers were assessed by enzyme linked immunosorbent assays (ELISA) for human EPO protein at 4- and 24-hours post-injection (FIG. 5B). Mouse fetuses injected with PBS as a control revealed that minimal, if any, EPO is present in the livers of fetuses without LNP treatment (FIG. 5B). Therefore, the presence of EPO indicates successful LNP and mRNA delivery to fetal livers. There was a clear dose-dependent response in EPO production following treatment with LNPs at both time points. This is exemplified by the 5-fold higher EPO expression at 4 hours in fetuses injected with the higher volume of pA-3.EPO compared with those injected with the lower volume (FIG. 5B). Further, fetal livers collected 24 hours post-injection had lower EPO content compared to those collected 4 hours post-injection, as was the case with luciferase mRNA delivery. Lastly, LNP pA-3.EPO yielded 2-fold higher EPO content than LNP B-4.EPO 4 hours after injection. These results strongly agree with our luciferase and GFP expression data, confirming that these LNP platforms are robust and can deliver several types of mRNA, including those that model protein replacement therapies to treat prenatal disease.

Quantification of Erythropoietin Production. The ionizable lipids used in LNP formulations A-3 and B-4 were purified as described above. LNPs were prepared with human EPO mRNA (TriLink Biotechnologies) and injected into E16 fetuses via the vitelline vein. Fetal livers were harvested at 4 hours and 24 hours post-injection and kept on ice during processing. Livers were rinsed three times with 1×PBS to remove excess blood and were then homogenized in 5 mL of 1×PBS using 15 mL tissue grinders (ThermoFisher). Lysates were brought through two repeated overnight freeze and thaw cycles to break up cell membranes. After the final thaw, processed liver samples were centrifuged (5 minutes, 5000×g) and the supernatant was collected for analysis. EPO content in the supernatant was quantified by enzyme-linked immunosorbent assay (ELISA) using the Human Erythropoietin Quantikine IVD ELISA Kit (R&D Systems, Minneapolis, MN) per manufacturer recommendations. Data shown are average and SEM of EPO concentrations from at least three fetuses injected in each experimental group.

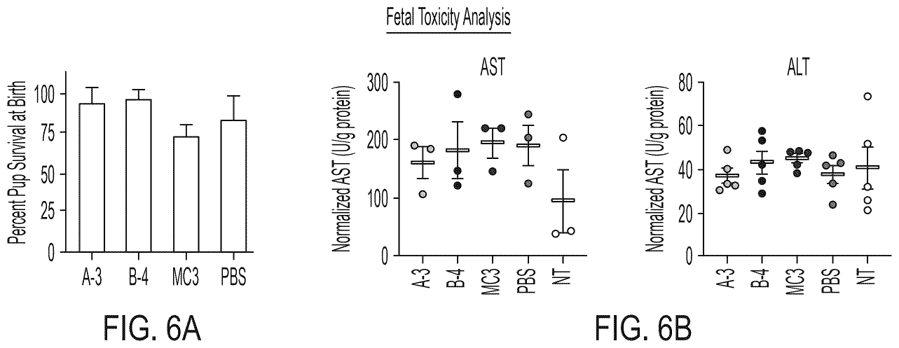

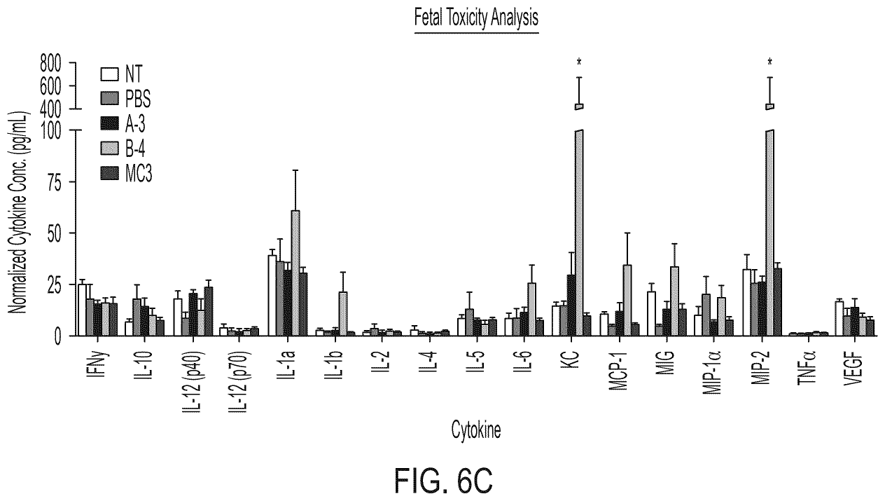

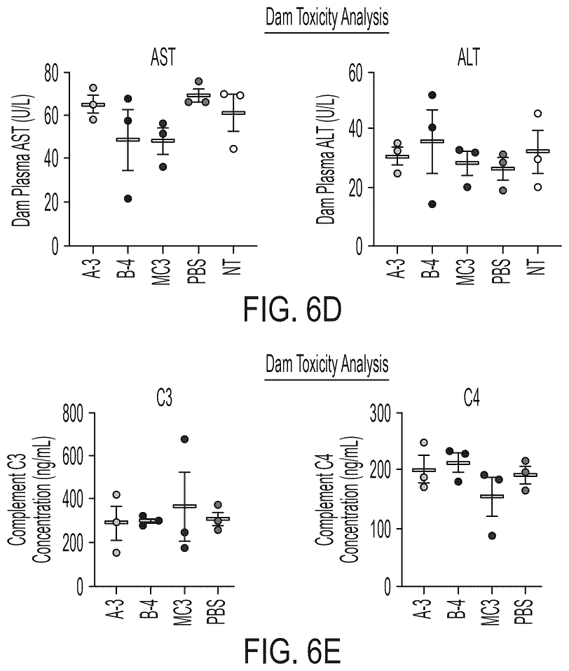

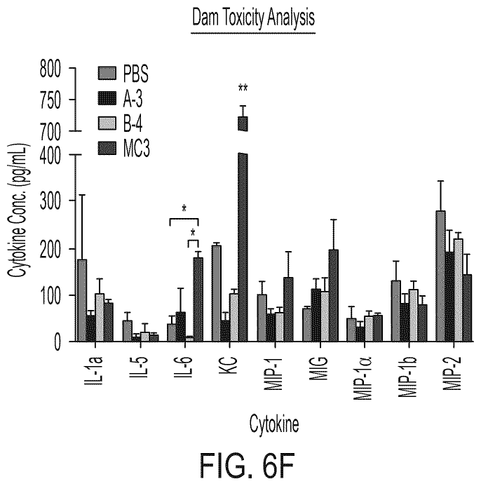

Example 7: LNPs are safe for Nucleic Acid Delivery to Fetuses and Do Not Induce Fetal Loss. Survival and toxicity were assessed at E19 following LNP injection at E16 to remove the variables of poor parenting and pup death related to the natural birthing process. As such, injected fetuses were delivered by cesarean section and assessed for gross appearance, presence of spontaneous movements and visible precordial palpations (heartbeat) to assess for survival at E19 (FIG. 6A, 11A). Balb/c fetuses injected with either LNP pA-3.luc or pB-4.luc had >90% survival which was comparable to control fetuses injected with PBS. Alternatively, fetuses injected with LNP MC3.luc had a survival rate of 72.4%. These results suggest that survival from in utero delivery of our LNPs (LNP pA-3.luc and pB-4.luc) is no different than that associated with the procedural related toxicity in the mouse model. Further, our results suggest that LNP MC3.luc may be more toxic to fetuses compared to our LNPs, although the difference in survival between each treatment group was not statistically significant. Survival following LNP injection in C57BL/6 fetuses was also evaluated to directly compare LNP toxicity in different strains of mice. All C57BL/6 treatment groups had 100% survival, indicating that prenatal delivery of our LNPs do not result in loss of viability in C57BL/6 mice, and there is minimal difference in survival between C57BL/6 and Balb/c strains (FIG. 11A).

Fetal Survival and Protein Extraction. LNPs A-3, B-4, or DLin-MC3-DMA, PBS, or LPS/GPC were injected into E16 fetuses via the vitelline vein. All the fetuses in each dam (n=3 dams) were injected. On E19, blood from the dams was collected retro-orbitally into blood collection tubes, centrifuged (10,000×g, 10 minutes, 4° C.), and plasma was harvested for cytokine analysis, liver enzymes, and complement activation. Next, fetuses were surgically removed to avoid any variability from the natural birthing process and poor parenting, and the pups were immediately evaluated for survival. Survival was assessed by observation of gross appearance, size, spontaneous movement, and a visible heartbeat. After assessing survival, pup livers were collected from a minimum of 5 injected fetuses for cytokine analysis and ALT/AST analysis (see below). The remaining injected fetuses were housed with foster female mice that had given birth to pups no more than one day prior to fostering.

Dam plasma and fetal livers were snap-frozen in dry ice and stored at −80° C. until use. Frozen livers were cut on dry ice, and pieces were weighed using a Mettler-Toledo scale, thawed in 5 mL/g of tissue M-Per™ lysis buffer (ThermoFisher) with Complete™ protease inhibitor cocktail (Roche, Basel, Switzerland) and immediately disrupted using the TissueLyzer II® (Qiagen, Inc., Germantown, MD) system with 5 mm steel beads (Qiagen, Inc.) under 30 Hz frequency for 60 seconds; the homogenization process was repeated twice. Homogenized tissues were placed on ice for 30 min to allow complete lysis. Samples were centrifuged (2200×g, 30 minutes, 4° C.) and the supernatant was collected into new tubes. Total protein content in each sample was determined using the microBCA protein assay kit (ThermoFisher) per manufacturer instructions.

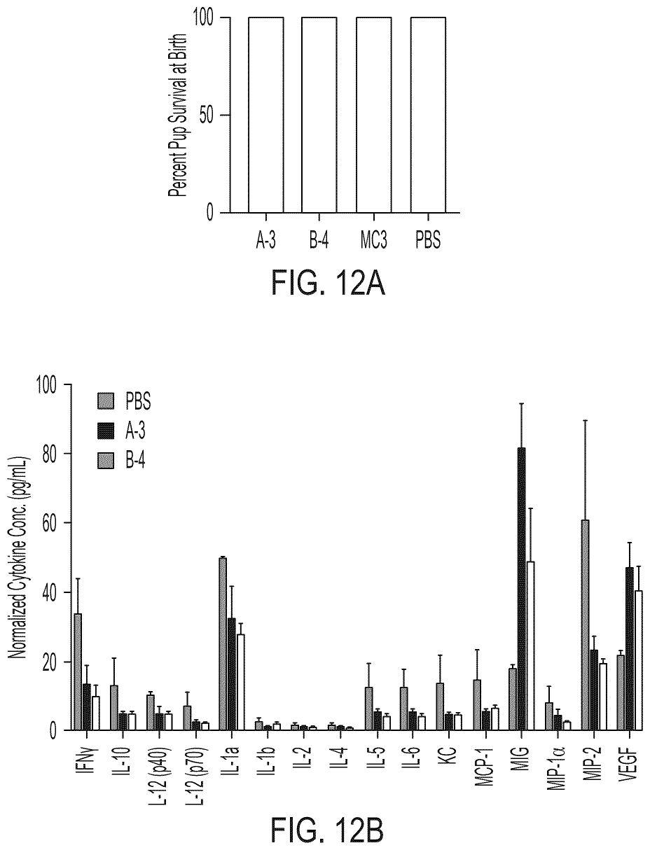

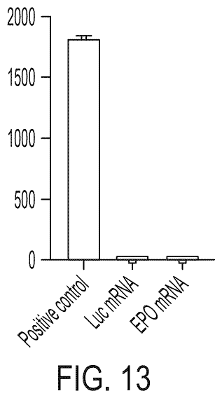

Example 8: LNP Injections Result in Minimal Fetal Immunotoxicity or Liver Damage. Given the high efficiency of liver accumulation of our LNPs, it was determined if prenatal LNP delivery resulted in liver toxicity or activation of an inflammatory response in the fetus or dam. First, to demonstrate that the mRNA used in this study is not independently immunogenic, human dendritic cells were transfected with luciferase mRNA or EPO mRNA and evaluated IFN-α expression levels in culture media after 24 hours. Following treatment, there was no increase in IFN-α levels, indicating that the mRNA itself is not immunogenic (FIGS. 12A and 12B).

Fetal liver toxicity following LNP injection was assessed by quantifying the liver enzymes alanine aminotransferase (ALT) and aspartate aminotransferase (AST) from fetal liver tissue at E19. There was a minor increase in AST levels from all LNP- and PBS-injected mice compared to untreated controls that was not statistically significant. However, AST levels following injection with our LNPs A-3.luc and B-4.luc were comparable to mice treated with either MC.3.luc or PBS. This indicates that the injection procedure itself may affect AST levels. Importantly, there were no significant changes in ALT or AST levels from fetal livers treated with our LNPs or controls, indicating that our LNPs enable nucleic acid delivery to fetal livers without inducing enzyme release associated toxicity at the timepoints evaluated here (FIG. 6B).

The induction of an inflammatory response to LNPs A-3.luc, B-4.luc, and MC3.luc was evaluated to further elucidate their safety for fetal therapy. Cytokine production from E19 livers of Balb/c fetuses that had been injected with LNPs A-3.luc, B-4.luc, MC3.luc, or PBS (FIG. 6C) was assessed. Most cytokine levels were not significantly different in LNP-treated fetuses compared to untreated or PBS-treated control fetuses. Importantly, no cytokines were elevated in response to LNP A-3.luc, our top performing LNP for mRNA delivery. Two chemokines, KC (also termed IL8/CXCL1) and MIP-2 (also termed CXCL2), were elevated in fetal livers in response to LNP B-4.luc. These two chemokines are potent neutrophil attractants and play a role in inflammation, wound healing, angiogenesis, and other biological processes (51). To evaluate any mouse strain-specific cytokine response to the LNPs, a similar analysis on E19 fetal livers harvested from C57BL/6 mice injected with LNPs A-3.luc, B-4.luc, MC3.luc, or PBS was conducted. This analysis did not reveal a similar increase in KC and MIP-2 levels as that found in Balb/c mice (FIG. 11B). Rather, treatment with our LNPs in C57BL/6 mice resulted in decreased MIP-2 expression and increased MIG and VEGF levels compared to PBS injections (FIG. 11B). These differences between Balb/c and C57BL/6 mice may be partly due to the fact that they develop different T cell mediated immune responses (52).

Analysis of mRNA and LNP Immunogenicity. Monocyte-derived human dendritic cells were used to evaluate the immunogenicity of mRNA without LNP carriers. Dendritic cells were plated at 200,000 cells/well. Luciferase or EPO mRNA were complexed with TransIT mRNA transfection reagent (MirusBio, Madison, WI) and added to hMD-DCs at 0.3 μg/well. The supernatant was collected 24 hours post-transfection and evaluated for human IFN-α by an enzyme-linked immunosorbent assay (Mabtech, 3425-1H-20, Cincinnati, OH) per manufacturer instructions.

For cytokine analysis in vivo, prepared fetal liver lysates and dam plasma samples were assessed for cytokine levels 3 days post LNP or PBS (control) injection using a 20 pro-inflammatory cytokine panel using Luminex® technology. Plates were developed using the Milliplex® assay builder (Millipore Sigma, Burlington, MA) and subjected to manufacturer's quality control. Individual plates were used to analyze dam plasma samples or fetal livers. A standard curve for each plate was prepared by serially diluting the Milliplex® Pro Mouse Cytokine Standard 20-Plex in the standard diluent (1:4 to 1:65,536). Plasma samples were diluted 1:20 and fetal liver samples were diluted 1:2 using the sample diluent, and 30 μL was transferred to the assay plates with prefilled capture antibodies. Each sample was plated in duplicate with 5 biological replicates per treatment group. The assay plates were incubated under orbital shaking (800 rpm) for 30 minutes, washed twice before the addition of biotinylated detection antibodies. Plates were incubated for 30 minutes (800 rpm), washed, and revealed with streptavidin-phycoerythrin for 10 min (800 rpm). Multiplex plates were run on the Bioplex 2200 system with a minimum of 50 beads analyzed per region. Each cytokine was assessed using a 5-parameter regression algorithm (5-PL), with samples from LPS/GPC injected mice as the positive control. Liver cytokine data was normalized to the protein concentration in the sample as determined by the microBCA assay.

Liver Toxicity and Complement System Analysis. For liver toxicity studies, plasma samples from dams or fetal liver lysates were assessed for AST and ALT liver enzyme levels using AST or ALT colorimetric activity assay kits, respectively (Cayman Chemicals, Ann Arbor, Michigan, USA) according to manufacturer recommendations at 3 days post LNP or PBS injection. Of note, due to the small fetal size and limited fetal serum availability, fetal cytokine production and fetal liver enzymes were assessed in fetal liver lysates rather than serum. Thus, fetal AST/ALT data was normalized to the protein concentration in the sample as determined by the microBCA assay. Complement system activation from plasma collected from the dams was assessed using Immunotag Mouse C3 (Complement C3) and Immunotag Mouse C4 (Complement C4) ELISA kits (G-Biosciences, St. Louis, Missouri, USA) per manufacturer instructions. Because of the low volume of blood present in fetuses at this gestational age, complement system activation directly from fetal plasma was unable to be assessed.

Example 9: Lack of Maternal Toxicity following in utero LNP Delivery. Any fetal intervention involves two patients, the fetus and the mother, that could be potentially affected by the treatment (53). Thus, the toxicity of prenatal LNP delivery on the pregnant dams were evaluated. No maternal deaths (both Balb/c and C57BL/6 dams) were noted following in utero LNP delivery throughout all the experiments. There were no differences in ALT and AST levels between Balb/c dams whose fetuses were injected with LNPs A-3.luc, B-4.luc, MC3.luc, or PBS (FIG. 6D), suggesting that in utero LNP delivery did not result in maternal liver toxicity (FIG. 6D). Maternal Balb/c plasma was also assessed for C3 and C4 levels to assess complement activation in the mothers of fetuses undergoing in utero LNP or PBS injection and no significant differences were noted between experimental and control mice (FIG. 6E). Lastly, analysis of cytokine levels from plasma collected from Balb/c dams at E19 revealed few significant changes between those injected with LNPs or PBS, and most cytokines tested were below the assay's detection level (FIG. 6F). Of note, KC and IL-6 were significantly upregulated in serum of dams with fetuses injected with LNP MC3.luc compared to other treatment groups (FIG. 6F). These results demonstrate that our LNPs do not induce liver damage, an inflammatory response, or activation of the complement system in the dams of injected fetuses.

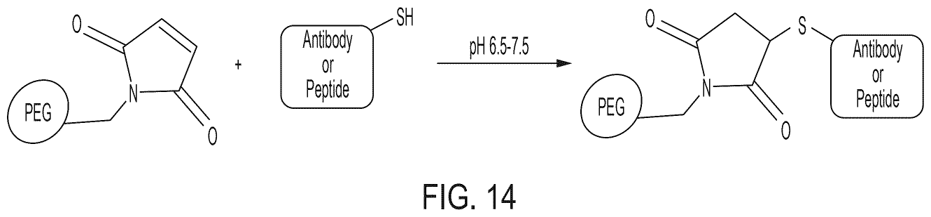

Example 10: Organ and Cell Nanoparticle Targeting. Many disease processes that are targets for nucleic acid therapeutics including, but not limited to, gene therapy, gene editing, enzyme replacement therapy and RNA therapeutics, involve specific organs. Depending on the disease, organs that are involved could be multiple of limited to a single organ. Furthermore, within an organ made up of multiple different cell types, specific cells may be involved in the disease process. For example, many genetic lung diseases result from pathology that effects the epithelial cells of the lung but not the pulmonary endothelial or mesenchymal cells. Thus, to increase the efficacy and specificity of therapeutics delivered before and/or after birth by lipid nanoparticles (LNP), the LNPs were modified to contain organ/cell targeting moieties. Specifically, thiol-maleimide chemistry was used to attach peptides and/or antibodies to the PEG component of the LNP such that the LNP can be targeted to the intended organ (FIG. 14). Specifically, the PEG to be used to form the LNP contains a maleimide group and the antibody or peptide contains a sulfhydryl group (also called thiol). When combined at a pH 6.5-7.5, a stable thioether nonreversible linkage is formed between the maleimide on the PEG and the thiol group on the peptide or antibody. The modified PEG is then combined with the LNP micelles to make an LNP containing the PEG-antibody or peptide conjugate to produce a targeting LNP.

Peptide and antibody conjugates have been devised to target the heart, brain, lung and hematopoietic cells including hematopoietic stem and progenitor cells (HSCs). These conjugates would be employed for the treatment of genetic diseases including, but not limited to, those listed in Table 1 below. Furthermore, the organ/cell targeting antibody and peptide are also listed in Table 2 below.

TABLE 1
Genetic diseases targeted for LNP mediated therapeutics.

| Disease | |
|---|---|
| Category | Disease |
| Hematologic | Sickle cell anemia, Beta-thalassemia, Alpha-thalassemia, |
| | Fanconi anemia |
| Neurodevel- | Krabbe disease, Lysosomal storage diseases including |
| opmental | MPSI and MPS VII, Neiman Pick disease, Freidreich's |
| | ataxia, Fragile X, Pompe disease |
| Lung disease | Cystic fibrosis, Surfactant deficiencies icluding surfactant |
| | protein B deficiency, surfactant protein C deficiency, |
| | ABCA3 deficiency, Alpha-1 antitrypsin deficiency |
| Heart disease | Hypertrophic Cardiomyopathy, Friedreich's ataxia, MPS IH |

TABLE 2
Moieties (peptides/antibodies) to enhance
LNP targeting to specific organs/cells.

| Target | Peptide/Antibody |
|---|---|
| Hematopoietic | anti cKit antibody |
| stem and | anti CD45 antibody |
| progenitor cells | anti CD34 antibody |
| | Stem cell factor peptide |
| | SDF-1 alpha peptide |
| | Thrombopoietin-memetic peptide-IEGPTLRQWLAARA |
| Brain | Transferrin receptior targeting peptid-CGGGHKYLRW |
| | Transferrin receptor antibody |
| | Peptide PB1-CTFYGGRPKRNNFLRGIR |
| Heart | Peptide PH1-APWHLSSQYSRT |
| Lung | Peptide SynL1-CPSSSREKC |
| | Peptide IntL1-CGACYGLPHKFCG |
| | Peptide EpCAML1-D-WRPTRURLLPWWICGSGSK |
| | Peptide EndothelinL1-CHLDIIW |
| | Peptde LegL1-CGACYGLPHKFCG |
| | anti EpCam antibody |
| | antiICAM-1 antibody |

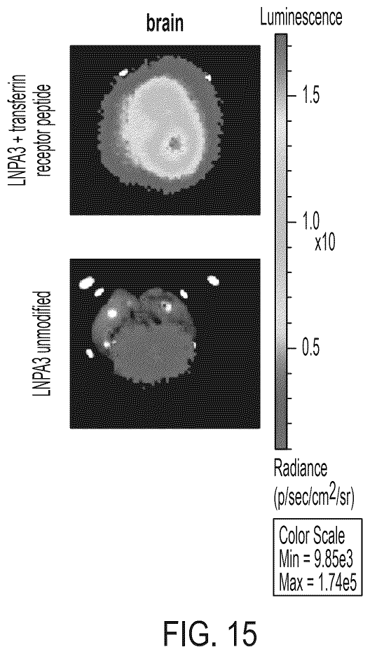

Studies testing this approach via conjugation of a transferring receptor targeting peptide to the PEG followed by its incorporation into a LNP demonstrated a 2-3-fold increase in brain targeting of the LNP in the postnatal mouse model. Specifically, the modified LNP or unmodified LNP containing luciferase mRNA was injected into day of life 2 mice intravascularly. Brains were assessed for luciferase expression 4 hours after injection. Those that received the modified LNP demonstrated increased luciferase expression (FIG. 15).

Finally, in order to increase the selection of targeted hematopoietic cells, LNPs that contain agents that improve the survival and proliferation of HSCs were devised. These agents will be incorporated into nontargeting LNPs as well as LNPs that contain PEGs modified with antibodies/peptides that target HSCs. These agents are listed in Table 3.

TABLE 3
Compounds incorporated into LNPs to enhance HSC survival.
Compounds to increase HSC survival

Prostaglandin e2
Diprotin A
LL-37

### REFERENCES

1. Y. M. D. Lo, M. S. C. Tein, T. K. Lau, C. J. Haines, T. N. Leung, P. M. K. Poon, J. S. Wainscoat, P. J. Johnson, A. M. Z. Chang, N. M. Hjelm, Quantitative Analysis of Fetal DNA in Maternal Plasma and Serum: Implications for Noninvasive Prenatal Diagnosis. Am. J. Hum. Genet. 62, 768-775 (1998).
2. D. W. Bianchi, J. M. Williams, L. M. Sullivan, F. W. Hanson, K. W. Klinger, A. P. Shuber, PCR Quantitation of Fetal Cells in Maternal Blood in Normal and Aneuploid Pregnancies. Am. J. Hum. Genet. 61, 822-829 (1997).
3. C. R. Ferreira, W. A. Gahl, Lysosomal storage diseases. Transl. Sci. Rare Dis. 2, 1-71 (2017).
4. G. Massaro, C. N. Z. Mattar, A. M. S. Wong, E. Sirka, S. M. K. Buckley, B. R. Herbert, S. Karlsson, D. P. Perocheau, D. Burke, S. Heales, A. Richard-londt, S. Brandner, M. Huebecker, D. A. Priestman, F. M. Platt, K. Mills, A. Biswas, J. D. Cooper, J. K. Y. Chan, S. H. Cheng, S. N. Waddington, A. A. Rahim, Fetal gene therapy for neurodegenerative disease of infants. Nat. Med. 24 (2018), doi: 10.1038/s41591-018-0106-7.
5. D. Alapati, W. J. Zacharias, H. A. Hartman, A. C. Rossidis, J. D. Stratigis, N. J. Ahn, B. Coons, S. Zhou, H. Li, K. Singh, J. Katzen, Y. Tomer, A. C. Chadwick, K. Musunuru, M. F. Beers, E. E. Morrisey, W. H. Peranteau, In utero gene editing for monogenic lung disease. Sci. Transl. Med. 11, 1-13 (2019).
6. S. DeWeerdt, Prenatal Gene Therapy Offers the Earliest Possible Cure. Nature (2018), (available at https://www.nature.com/articles/d41586-018-07643-z).
7. G. Almeida-porada, A. Atala, C. D. Porada, In utero stem cell transplantation and gene therapy: rationale, history, and recent advances toward clinical application. Mol. Ther. Clin. Dev. 5 (2016), doi: 10.1038/mtm.2016.20.
8. D. Songdej, C. Babbs, D. Higgs, An international registry of survivors with Hb Bart's hydrops fetalis syndrome. Blood. 129, 1251-1259 (2017).
9. D. Chui, J. Waye, Hydrops fetalis caused by alpha-thalassemia: an emerging health care problem. Blood. 91, 2213-2222 (1998).
10. B. Connolly, C. Isaacs, L. Cheng, K. H. Asrani, R. R. Subramanian, SERPINA1 mRNA as a Treatment for Alpha-1 Antitrypsin Deficiency. J. Nucleic Acids. 2018, 1-7 (2018).
11. J. F. Apgar, J. Tang, P. Singh, N. Balasubramanian, J. Burke, M. R. Hodges, M. A. Lasaro, L. Lin, B. L. Miliard, K. Moore, L. S. Jun, S. Sobolov, A. K. Wilkins, X. Gao, Quantitative Systems Pharmacology Model of hUGT1A1-modRNA Encoding for the UGT1A1 Enzyme to Treat Crigler-Najjar Syndrome Type 1. CPT Pharmacometrics Syst. Pharmacol. 7, 404-412 (2018).
12. D. S. Roseman, T. Khan, F. Rajas, L. S. Jun, K. H. Asrani, C. Isaacs, J. D. Farelli, R. R. Subramanian, G6PC mRNA Therapy Positively Regulates Fasting Blood Glucose and Decreases Liver Abnormalities in a Mouse Model of Glycogen Storage Disease 1a. Mol. Ther. 26, 814-821 (2018).
13. F. Derosa, B. Guild, S. Karve, L. Smith, K. Love, J. R. Dorkin, K. J. Kauffman, J. Zhang, B. Yahalom, D. G. Anderson, Therapeutic efficacy in a hemophilia B model using a biosynthetic mRNA liver depot system. Gene Ther. 23, 699-707 (2016).
14. Z. Trepotec, E. Lichtenegger, C. Plank, M. K. Aneja, C. Rudolph, Delivery of mRNA Therapeutics for the Treatment of Hepatic Diseases. Mol. Ther. 27, 794-802 (2019).
15. A. S. Ricciardi, R. Bahal, J. S. Farrelly, E. Quijano, A. H. Bianchi, V. L. Luks, R. Putman, F. López-giráldez, S. Co, E. Song, Y. Liu, W. Hsieh, D. H. Ly, D. H. Stitelman, P. M. Glazer, W. M. Saltzman, In utero nanoparticle delivery for site-specific genome editing. Nat. Commun. 9 (2018), doi: 10.1038/s41467-018-04894-2.
16. A. C. Rossidis, J. D. Stratigis, A. C. Chadwick, H. A. Hartman, N. J. Ahn, H. Li, K. Singh, B. E. Coons, L. Li, W. Lv, W. Zoltick, D. Alapati, W. Zacharias, R. Jain, E. E. Morrisey, K. Musunuru, W. H. Peranteau, In utero CRISPR-mediated therapeutic editing of metabolic genes. Nat. Me. 24, 1513-1518 (2018).
17. M. G. Davey, J. S. Riley, A. Andrews, A. Tyminski, M. Limberis, J. E. Pogoriler, E. Partridge, A. Olive, H. L. Hedrick, A. W. Flake, W. H. Peranteau, Induction of Immune Tolerance to Foreign Protein via Adeno-Associated Viral Vector Gene Transfer in Mid-Gestation Fetal Sheep. PLos One. 12, 1-17 (2017).
18. N. Pardi, M. J. Hogan, F. W. Porter, D. Weissman, mRNA vaccines—a new era in vaccinology. Nat. Rev. Drug Discov. 17, 261-279 (2018).
19. R. S. Riley, C. H. June, R. Langer, M. J. Mitchell, Delivery technologies for cancer immunotherapy. Nat. Rev. Drug Discov. 18, 175-196 (2019).
20. K. A. Hajj, K. A. Whitehead, Tools for translation: Non-viral materials for therapeutic mRNA delivery. Nat. Rev. Mater. 2, 1-17 (2017).
21. P. P. G. Guimaraes, R. Zhang, R. Spektor, M. Tan, A. Chung, M. M. Billingsley, R. El-mayta, R. S. Riley, L. Wang, J. M. Wilson, M. J. Mitchell, lonizable lipid nanoparticles encapsulating barcoded mRNA for accelerated in vivo delivery screening. J. Control. Release. 316, 404-417 (2019).
22. O. S. Fenton, K. N. Olafson, P. S. Pillai, M. Mitchell, R. Langer, Advances in Biomaterials for Drug Delivery. Adv. Mater. 30 (2018).
23. L. E. Mcclain, A. W. Flake, In utero stem cell transplantation and gene therapy: Recent progress and the potential for clinical application. Best Pract. Res. Clin. Obstet. Gynaecol. 31, 88-98 (2016).
24. J. L. Roybal, M. T. Santore, A. W. Flake, Seminars in Fetal & Neonatal Medicine Stem cell and genetic therapies for the fetus. Semin. Fetal Neonatal Med. 15, 46-51 (2010).
25. R. Karda, S. M. K. Buckley, C. N. Mattar, J. Ng, G. Massaro, M. A. Kurian, J. Baruteau, P. Gissen, J. K. Y. Chan, C. Bacchelli, S. N. Waddington, A. A. Rahim, M. R. Haddad, Perinatal systemic gene delivery using adeno-associated viral vectors. 7, 1-6 (2014).
26. S. J. Howe, M. R. Mansour, K. Schwarzwaelder, C. Bartholomae, M. Hubank, H. Kempski, M. H. Brugman, K. Pike-overzet, S. J. Chatters, D. De Ridder, K. C. Gilmour, S. Adams, S. I. Thornhill, K. L. Parsley, F. J. T. Staal, R. E. Gale, D. C. Linch, J. Bayford, L. Brown, M. Quaye, C. Kinnon, P. Ancliff, D. K. Webb, M. Schmidt, C. Von Kalle, H. B. Gaspar, A. J. Thrasher, Insertional mutagenesis combined with acquired somatic mutations causes leukemogenesis following gene therapy of SCID-X1 patients. 118 (2008), doi: 10.1172/JCI35798DS1.
27. A. J. Mukalel, R. S. Riley, R. Zhang, M. J. Mitchell, Nanoparticles for nucleic acid delivery: Applications in cancer immunotherapy. Cancer Lett. 458, 102-112 (2019).
28. S. B. Fournier, J. N. D. Errico, P. A. Stapleton, Engineered nanomaterial applications in perinatal therapeutics. Pharmacol. Res. 130, 36-43 (2018).
29. F. Alexis, E. Pridgen, L. K. Molnar, O. C. Farokhzad, Factors Affecting the Clearance and Biodistribution of Polymeric Nanoparticles. Mol. Pharm. 5, 505-515 (2008).
30. F. Danhier, E. Ansorena, J. M. Silva, R. Coco, A. Le Breton, V. Préat, PLGA-based nanoparticles: An overview of biomedical applications. J. Control. Release. 161, 505-522 (2012).
31. H. K. Mandl, E. Quijano, H. W. Suh, E. Sparago, S. Oeck, M. Grun, P. M. Glazer, W. M. Saltzman, Optimizing biodegradable nanoparticle size for tissue-specific delivery. J. Control. Release. 314, 92-101 (2019).
32. W. Huang, C. Zhang, Tuning the size of poly(lactic-co-glycolic acid) (PLGA) nanoparticles fabricated by nanoprecipitation. Biotechnol. J. 13 (2018).
33. M. C. Operti, Y. Dolen, J. Keulen, E. A. van Dinther, C. G. Figdor, O. Tagit, Microfluidics-Assisted Size Tuning and Biological Evaluation of PLGA particles. Pharmaceutics. 11 (2019).
34. K. J. Kauffman, J. R. Dorkin, J. H. Yang, M. W. Heartlein, F. Derosa, F. F. Mir, O. S. Fenton, D. G. Anderson, Optimization of Lipid Nanoparticle Formulations for mRNA Delivery in Vivo with Fractional Factorial and Definitive Screening Designs. Nano Lett. 15, 7300-7306 (2015).
35. M. A. Oberli, A. M. Reichmuth, J. R. Dorkin, M. J. Mitchell, O. S. Fenton, A. Jaklenec, D. G. Anderson, R. Langer, D. Blankschtein, Lipid Nanoparticle Assisted mRNA Delivery for Potent Cancer Immunotherapy. Nano Lett. 17, 1326-1335 (2017).
36. K. A. Hajj, R. L. Ball, S. B. Deluty, S. R. Singh, D. Strelkova, C. M. Knapp, K. A. Whitehead, Branched-Tail Lipid Nanoparticles Potently Deliver mRNA In Vivo due to Enhanced Ionization at Endosomal pH. 1805097, 1-7 (2019).
37. T. M. Allen, P. R. Cullis, Liposomal drug delivery systems: From concept to clinical applications ⋆. Adv. Drug Deliv. Rev. 65, 36-48 (2013).
38. I. Zuhorn, U. Bakowsky, E. Polushkin, W. Visser, M. Stuart, J. Engberts, D. Hoekstra, Nonbilayer Phase of Lipoplex-Membrane Mixture Determines Endosomal Escape of Genetic Cargo and Transfection Efficiency. Mol. Ther. 11, 801-810 (2005).
39. D. Chen, K. T. Love, Y. Chen, A. A. Eltoukhy, C. Kastrup, G. Sahay, A. Jeon, Y. Dong, K. A. Whitehead, D. G. Anderson, Rapid discovery of potent siRNA-containing lipid nanoparticles enabled by controlled microfluidic formulation. J. Am. Chem. Soc. 134, 6948-6951 (2012).
40. K. T. Love, K. P. Mahon, G. Christopher, K. A. Whitehead, W. Querbes, J. Robert, J. Qin, W. Cantley, L. L. Qin, M. Frank-kamenetsky, K. N. Yip, R. Alvarez, D. W. Y. Sah, A. De Fougerolles, K. Fitzgerald, V. Koteliansky, A. Akinc, R. Langer, G. Daniel, K. T. Love, K. P. Mahon, C. G. Levins, K. A. Whitehead, W. Querbes, J. Robert, Lipid-like materials for low-dose, in vivo gene silencing. Proc. Natl. Acad. Sci. 107, 9915-9915 (2010).
41. M. Jayaraman, S. M. Ansell, B. L. Mui, Y. K. Tam, J. Chen, X. Du, D. Butler, L. Eltepu, S. Matsuda, J. K. Narayanannair, K. G. Rajeev, I. M. Hafez, A. Akinc, M. A. Maier, M. A. Tracy, P. R. Cullis, T. D. Madden, M. Manoharan, M. J. Hope, Maximizing the potency of siRNA lipid nanoparticles for hepatic gene silencing in vivo. Angew. Chemie-Int. Ed. 51, 8529-8533 (2012).
42. K. A. Whitehead, J. R. Dorkin, A. J. Vegas, P. H. Chang, O. Veiseh, J. Matthews, O. S. Fenton, Y. Zhang, K. T. Olejnik, V. Yesilyurt, D. Chen, S. Barros, B. Klebanov, T. Novobrantseva, R. Langer, D. G. Anderson, Degradable lipid nanoparticles with predictable in vivo siRNA delivery activity. Nat. Commun. 5, 1-10 (2014).
43. O. M. Swartley, J. F. Foley, D. P. Livingston, J. M. Cullen, S. A. Elmore, Histology Atlas of the Developing Mouse Hepatobiliary Hemolymphatic Vascular System with Emphasis on Embryonic Days 11.5-18.5 and Early Postnatal Development. Toxicol. Pathol. 44, 705-725 (2016).
44. S. M. Sarett, T. A. Werfel, L. Lee, M. A. Jackson, K. V Kilchrist, D. Brantley-sieders, C. L. Duvall, Lipophilic siRNA targets albumin in situ and promotes bioavailability, tumor penetration, and carrier-free gene silencing. PNAS. 114, E6490-E6497 (2017).
45. K. Garber, Alnylam launches era of RNAi drugs. Nat. Biotechnol. 36 (2018).
46. D. Adams, A. Gonzalez-Duarte, W. D. O'Riordan, C. C. Yang, M. Ueda, A. V. Kristen, I. Tournev, H. H. Schmidt, T. Coelho, J. L. Berk, K. P. Lin, G. Vita, S. Attarian, V. Planté-Bordeneuve, M. M. Mezei, J. M. Campistol, J. Buades, T. H. Brannagan, B. J. Kim, J. Oh, Y. Parman, Y. Sekijima, P. N. Hawkins, S. D. Solomon, M. Polydefkis, P. J. Dyck, P. J. Gandhi, S. Goyal, J. Chen, A. L. Strahs, S. V. Nochur, M. T. Sweetser, P. P. Garg, A. K. Vaishnaw, J. A. Gollob, O. B. Suhr, Patisiran, an RNAi therapeutic, for hereditary transthyretin amyloidosis. N. Engl. J. Med. 379, 11-21 (2018).
47. X. Zhang, V. Goel, G. J. Robbie, Pharmacokinetics of Patisiran, the First Approved RNA Interference Therapy in Patients With Hereditary Transthyretin-Mediated Amyloidosis. J. Clin. Pharmacol. (2019), doi: 10.1002/jcph.1553.
48. A. A. Sidi, P. Ohana, S. Benjamin, M. Shalev, J. H. Ransom, D. Lamm, A. Hochberg, I. Leibovitch, Phase I/II Marker Lesion Study of Intravesical BC-819 DNA Plasmid in H19 Over Expressing Superficial Bladder Cancer Refractory to Bacillus Calmette-Guerin. JURO. 180, 2379-2383 (2008).
49. K. Bahl, J. J. Senn, O. Yuzhakov, A. Bulychev, L. A. Brito, K. J. Hassett, M. E. Laska, M. Smith, Ö. Almarsson, J. Thompson, A. M. Ribeiro, M. Watson, T. Zaks, G. Ciaramella, Preclinical and Clinical Demonstration of Immunogenicity by mRNA Vaccines against H10N8 and H7N9 Influenza Viruses. Mol. Ther. 25, 1316-1327 (2017).
50. O. F. Khan, E. W. Zaia, S. Jhunjhunwala, W. Xue, W. Cai, D. S. Yun, C. M. Barnes, J. E. Dahlman, Y. Dong, J. M. Pelet, M. J. Webber, J. K. Tsosie, T. E. Jacks, R. Langer, D. G. Anderson, Dendrimer-Inspired Nanomaterials for the in Vivo Delivery of siRNA to Lung Vasculature. Nano Lett. 15, 3008-3016 (2015).
51. K. De Filippo, R. B. Henderson, M. Laschinger, N. Hogg, Neutrophil Chemokines KC and Macrophage-Inflammatory Protein-2 Are Newly Synthesized by Tissue Macrophages Using Distinct TLR Signaling Pathways. J. Immunol. 180, 4308-4315 (2008).
52. H. Watanabe, K. Numata, T. Ito, K. Takagi, A. Matsukawa, Innate Immune Response in Th1- And Th2-dominant Mouse Strains. Shock. 22, 460-466 (2004).
53. S. M. Winkler, M. R. Harrison, P. B. Messersmith, Biomaterials in fetal surgery. Biomater. Sci. 7, 3092-3109 (2019).
54. B. Rink, J. R. B. Jr, D. N. S. Jr, Carrier Screening in the Age of Genomic Medicine. Obstet. Gynecol. 129, 35-40 (2017).
55. A. Safary, M. A. Khiavi, R. Mousavi, J. Barar, M. A. Rafi, Enzyme replacement therapies: what is the best option? BioImpacts. 8, 153-157 (2018).
56. J. A. Gorzelany, M. P. De Souza, Protein Replacement Therapies for Rare Diseases: A Breeze for Regulatory Approval? Sci. Transl. Med. 5, 1-4 (2013).
57. S. Ramaswamy, N. Tonnu, K. Tachikawa, P. Limphong, J. B. Vega, P. P. Karmali, Systemic delivery of factor IX messenger RNA for protein replacement therapy. Proc. Natl. Acad. Sci. 114, E1941-E1950 (2017).
58. B. Truong, G. Allegri, X. Liu, K. E. Burke, X. Zhu, S. D. Cederbaum, Lipid nanoparticle-targeted mRNA therapy as a treatment for the inherited metabolic liver disorder arginase deficiency. Proc. Natl. Acad. Sci. 116, 21150-21159 (2019).
59. C. P. Gheorghe, N. Boring, L. Mann, R. Donepudi, S. M. Lopez, S. P. Chauhan, V. Bhandari, M. J. K. J, A. Johnson, R. Papanna, Neonatal Outcomes and Maternal Characteristics in Monochorionic Diamniotic Twin Pregnancies: Uncomplicated versus Twin-to-Twin Transfusion Syndrome Survivors after Fetoscopic Laser Surgery. Fetal Diagn. Ther., 1-6 (2019).
60. M. L. Kovler, E. B. Jelin, Fetal intervention for congenital diaphragmatic hernia. Semin. Pediatr. Surg. 28, 150818 (2019).
61. N. S. Adzick, E. A. Thom, C. Y. Spong, J. W. Brock, P. K. Burrows, M. P. Johnson, L. J. Howell, J. A. Farrell, M. E. Dabrowiak, L. N. Sutton, N. Gupta, D. Ph, N. B. Tulipan, M. E. D. Alton, D. L. Farmer, A Randomized Trial of Prenatal versus Postnatal Repair of Myelomeningocele. N. Engl. J. Med. 364, 993-1004 (2011).
62. S. Montazersaheb, Potential of Peptide Nucleic Acids in Future Therapeutic Applications. Adv. Pharm. Bull. 8, 551-563 (2018).
63. M. M. Boelig, A. G. Kim, J. D. Stratigis, L. E. Mcclain, H. Li, A. W. Flake, W. H. Peranteau, Biology of Blood and Marrow Transplantation The Intravenous Route of Injection Optimizes Engraftment and Survival in the Murine Model of In Utero Hematopoietic Cell Transplantation. Biol. Blood Marrow Transplant. 22, 991-999 (2016).
64. P. Shangaris, S. P. Loukogeorgakis, S. Subramaniam, C. Flouri, L. H. Jackson, W. Wang, M. P. Blundell, S. Liu, S. Eaton, N. Bakhamis, D. L. Ramachandra, P. Maghsoudlou, L. Urbani, S. N. Waddington, A. Eddaoudi, J. Archer, M. N. Antoniou, D. J. Stuckey, M. Schmidt, A. J. Thrasher, T. M. Ryan, P. De Coppi, A. L. David, In Utero Gene Therapy (IUGT) Using GLOBE Lentiviral Vector Phenotypically Corrects the Heterozygous Humanised Mouse Model and Its Progress Can Be Monitored Using MRI Techniques. Sci. Rep. 9 (2019), doi: 10.1038/s41598-019-48078-4.
65. C. C. Qin, Y. N. Liu, Y. Hu, Y. Yang, Z. Chen, Macrophage inflammatory protein-2 as mediator of inflammation in acute liver injury. World J. Gastroenterol. 23, 3043-3052 (2017).
66. M. Sedic, J. J. Senn, A. Lynn, M. Laska, M. Smith, S. J. Platz, J. Bolen, S. Hoge, A. Bulychev, E. Jacquinet, V. Bartlett, P. F. Smith, Safety Evaluation of Lipid Nanoparticle-Formulated Modified mRNA in the SpragueDawley Rat and Cynomolgus Monkey. Vet. Pathol. 55, 341-354 (2018).
67. S. Sabnis, E. S. Kumarasinghe, T. Salerno, C. Mihai, T. Ketova, J. J. Senn, A. Lynn, A. Bulychev, I. McFadyen, J. Chan, O. Almarsson, M. G. Stanton, K. E. Benenato, A Novel Amino Lipid Series for mRNA Delivery: Improved Endosomal Escape and Sustained Pharmacology and Safety in Non-human Primates. Mol. Ther. 26, 1509-1519 (2018).
68. J. A. Kulkarni, P. R. Cullis, R. van der Meel, Lipid nanoparticles enabling gene therapies: from concepts to clinical utility. Nucleic Acid Ther. 28 (2018).
69. E. M. Kreger, S. T. Singer, R. G. Witt, N. Sweeters, B. Lianoglou, A. Lal, T. C. Mackenzie, E. Vichinsky, Favorable outcomes after in utero transfusion in fetuses with alpha thalassemia major: a case series and review of the literature. Prenat. Diagn. 36, 1242-1249 (2016).
70. J. Heyes, L. Palmer, K. Bremner, I. Maclachlan, Cationic lipid saturation influences intracellular delivery of encapsulated nucleic acids. 107, 276-287 (2005).
71. N. Ahn, J. Stratigis, B. Coons, A. Flake, H. Nah-Cederquist, W. Peranteau, Intravenous and Intra-amniotic In Utero Transplantation in the Murine Model. J Vis Exp. 140 (2018).

1. A method for the delivery of prenatal therapeutics, enzyme replacement therapy, or gene therapy to a fetus in need thereof, comprising introducing ionizable lipid nanoparticles (LNPs) nanoparticles comprising a therapeutic mRNA composition into the circulation of the fetus in need of treatment, wherein the ionizable LNPs deliver the therapeutic mRNA composition.

2. The method of claim 1, wherein the ionizable LNPs comprise:
one or more ionizable polyamine-lipids;
cholesterol;
1,2-Dioleoyl-sn-glycero-3-phosphoethanolamine (DOPE); and
a pegylated lipid (PEG-lipid).

3. The method of claim 2, wherein the one or more ionizable polyamine-lipids is selected from the group consisting of formulations: A-1 through A-5, B-1 through B-5, and C-1 through C-4 as described in FIGS. 2A and 2C.

4. The method of claim 2, wherein the PEG-Lipid is 1,2-dimyristoyl-snglycero-3-phosphoethanolamine-N-[methoxy (polyethylene glycol)-2000 (C14-PEG2000).

5. The method of claim 4, wherein the one or more ionizable polyamine lipids, cholesterol, DOPE, and C14-PEG2000 are at respective molar ratios of about 35:46.5:16:2.5.

6. The method of claim 1, wherein the mean size of the LNPs is about 60-140 nm.

7. The method of claim 1, wherein the encapsulation efficiency of mRNA is from about 70% to 100%.

8. The method of claim 1, wherein the pKa of the delivered ionizable LNPs is about 5.5-7.

9. The method of claim 1, wherein the nanoparticles are introduced into the circulation of a fetus intravenously.

10. The method of claim 1, wherein the nanoparticles are modified with a delivery target-specific antibody-conjugated PEG.

11. The method of claim 10, wherein the delivery target is expressed on the surface of an organ cell.

12. The method of claim 1, wherein the nanoparticles are modified with peptide-conjugated PEG to target a specific organ.

13. The method of claim 1, wherein the nanoparticles are modified with compounds to improve survival and proliferation of hematopoietic steam and progenitor cells (HSCs).

14. The method of claim 13, wherein the compounds to improve survival and proliferation of HSCs is selected from the group consisting of: prostaglandin e2, diprotin A, and LL-37.
